(19) United States
(12) Patent Application Publication
(10) Pub. No.: US 2011/0166428 A1
(43) Pub. Date: Jul. 7, 2011

(54) NOVEL METHOD FOR MEASURING INSULIN RESISTANCE

(75) Inventors: Catherine Ghezzi, Grenoble (FR); Daniel Fagret, Grenoble (FR); Jacques Demongeot, Sassenage (FR); Pascale Perret, Grenoble (FR)

(73) Assignees: UNIVERSITE JOSEPH FOURIER, GRENOBLE CEDEX 09 (FR); I.N.S.E.R.M. (Institut National de la Sante et de la Recherche Medicale), PARIS CEDEX 13 (FR)

(21) Appl. No.: 13/054,693

(22) PCT Filed: Jul. 17, 2009
(86) PCT No.: PCT/FR2009/051436

§ 371 Date: Mar. 18, 2011

(30) Foreign Application Priority Data
Jul. 18, 2008 (FR) 0804124

(51) Int. Cl. A61B 5/00

(52) U.S. Cl. 600/300

(57) ABSTRACT

Method of using at least one glucose derivative, halogenated in the 6 position, for the implementation of a process for the determination of insulin resistance in a mammal, in particular man, by measuring
the variation in the quantity (as a function of time) of the derivative in muscle cells during a given period t, after administration of the derivative, and
the variation in quantity (as a function of time) of the derivative in the muscle cells during a period essentially equal to the period t, after administration of the derivative, preceded by an administration of insulin.

The invention relates to a novel process for measurement of resistance to insulin.

Insulin resistance, characterized by a decrease in sensitivity to insulin in the insulin sensitive organs is one of the key elements of metabolic syndrome [Reaven G M. et al., Diabetes 1988, 37, 1595-1607]. This syndrome is characterized by central obesity, hypertension, glucose regulation abnormality and dyslipidaemia with elevated triglycerides and low levels of HDL.

Whatever the criteria may be for definition of this syndrome (WHO, National Cholesterol Education Program), its incidence reaches 15% in Europe and 23.7% in the United States. In non-diabetic persons, the presence of this syndrome results in an increase in mortality, all causes combined, one of the major causes being cardiovascular diseases. In type 2 diabetic patients, the presence of a metabolic syndrome results in an increase in the risk of cardiovascular events. It has been proved that the incidence of metabolic syndrome will increase in the near future, as a consequence of the increase in the incidence of diabetes and obesity [Zimmet P. et al., Nature, 2001, 414, 782-787].

The only method for direct measurement of insulin resistance, which is the reference technique, is the euglycaemic hyperinsulinaemic clamp. However, the clamp necessitates complex protocols which are restricting for the patient and cannot be envisaged in routine clinical practice.

Other methods have been proposed for determining insulin sensitivity in man indirectly, or by means of a substitution index, [Radzuik J. et al., J. Clin. End. Metab., 2000. 85(12), 4426-4433]. Some of them, the most simple, but also the least informative, are performed in the basal state and use measurements of glycaemia and insulinemia (HOMA, MINIMOD). More complex in vivo techniques enabling the study of the metabolism and transport of glucose in man in a given organ have been proposed, utilising nuclear magnetic resonance spectroscopy and [¹³C]-glucose [Rothman D L et al., Proc. Natl. Acad. Sci. USA, 1995, 92, 983-987], positron emission tomography (PET) and 2-[¹⁸F]-2-deoxyglucose (FDG) [Kelley D-E et al., J. Clin. Invest., 1996, 97, 2705-2713], or else tracer dilution techniques utilising [¹⁴C]-3-O-methyl-glucose (3OMG) [Bonadonna A et al., J. Clin. Invest., 1993, 92, 486-494], reference tracer for glucose transport.

However, none of these methods has found everyday clinical application owing to the difficulty of implementing these techniques.

Recently, decision trees taking into account of a group of risk factors associated with the HOMA test have been proposed for estimating the insulin resistance of a patient [Stern S E. et al., Diabetes, 2005, 54, 333-339]. However, this very inclusive approach finds its limits in the determination of the risk factors considered.

While there are currently discussions as to the criteria to take into account for each component of metabolic syndrome or as to the threshold values of these criteria, the scientific community is unanimous on the fact that there is no simple method, utilisable in the clinic, for measuring insulin resistance, the key element of metabolic syndrome. The development of such a technique can be regarded as a challenge to health care technologies.

An analogue of glucose, labelled with iodine 123, 6-Deoxy-6-Iodo-D-Glucose (6DIG) has been developed and validated as a pure tracer of glucose transport [Bignan G. et al., Patent FR2733753; Henry C. et al., Nucl. Med. Biol. 1997, 24, 527-534; Henry C. et al., Nucl. Med. Biol. 1997, 24, 99-104]. 6DIG has been used for evaluation of glucose transport, with a euglycaemic hyperinsulinic clamp, in rats fed with fructose [Perret P. et al., Eur. J. Nucl. Med. Mol. Imaging, 2007, 34(5), 734-744]. Moreover, 6DIG has also been used in the context of a method for the determination of cardiac insulin resistance in the rat using a NaI probe.

Cardiologists and diabetologists are interested in insulin resistance from different points of view. There is no simple and reliable method, useable in the clinic without excessive constraint on the patient, and which makes it possible to determine insulin resistance in different organs simultaneously, and thus to provide data pertinent both for cardiologists and diabetologists.

One of the purposes of the invention is to provide a method for the determination of insulin resistance.

One of the other aspects of the invention is to provide a simple and reliable method, useable in the clinic, for the determination of insulin resistance.

One of the other aspects of the invention is to provide a method making it possible to determine insulin resistance on the basis of several organs simultaneously.

The present invention relates to the use of at least one derivative of glucose, halogenated in the 6 position, for the implementation, by means of γ radiation detection, of a process for the determination of insulin resistance in a mammal, in particular man, by measurement
on the one hand of the variation in the quantity (as a function of time) of the aforesaid derivative in muscle cells during a given period Δt, after administration of the aforesaid derivative, and
on the other hand of the variation in quantity (as a function of time) of the aforesaid derivative in the aforesaid muscle cells during a period essentially equal to the aforesaid period Δt, after administration of the aforesaid derivative, preceded by an administration of insulin.

The present invention relates to the use of at least one derivative of glucose, halogenated in the 6 position, serving as a marker of glucose transport, for the implementation of a process for the determination of insulin resistance in a mammal, in particular man.

It is observed that the glucose exchanges within an organism can be followed through the incorporation of a marker, and that it is possible to observe a variation in the glucose exchanges following an injection of insulin. This variation in the exchanges can be linked to a disequilibrium of the glucose metabolism which can be due to an insulin resistance the measurement of which can, in combination with a clinical examination, assist in the diagnosis of a pathology involving insulin resistance. This insulin resistance, taken independently of any other element, cannot lead to a diagnosis owing to the fact that many disorders can be linked to insulin resistance. Only a medical professional is in a position to associate this disorder of the metabolism with other clinical elements and to determine from what pathology the patient is suffering.

The glucose exchanges observed correspond to the variations, in the course of time, in the quantity of marker, or tracer, administered, in different fluids or tissues of the organism. These fluids and tissues are likened to compartments, and these compartments are called “tissue” compartments if they represent tissues. The compartments can thus represent organs, muscles or fluids such as blood or the interstitial medium.

The first series of measurements makes it possible to obtain values indicating the glucose exchanges between the cells observed and their environment (for example the interstitial medium, or the blood), under conditions where the glucose metabolism is not modified by external factors. This first series of values is referred to as “basal”.

The second series of measurements makes it possible to obtain values indicating the glucose exchanges between the cells observed and their environment (for example the interstitial medium or the blood), under conditions where the glucose metabolism is modified by addition of insulin; this second series of values is referred to as “insulin”.

The addition of insulin has in particular the effect of stimulating the transport of glucose within the organism, favouring the entry of sugar into the insulin-dependent cells, which include the muscle cells [Cheatham B and Kahn C R, Endocrine Rev., 1995, 16, 117-142].

The insulin (Actrapide®) is administered at doses ranging from 2.5 to 3 IU/kg for animals. It is a rapid-acting insulin for intravenous injection.

By “derivative of glucose halogenated in the 6 position” is meant a molecule of glucose, in particular D-glucose, bearing an atom of halogen, in particular iodine or fluorine, on the 6 carbon of the glucose; the 6 carbon corresponds to the carbon bearing a primary alcohol when the glucose is in the pyranose form.

The doses of glucose derivative halogenated in the 6 position injected in the course of a protocol in the rat are from 0.1 to 10 mCi/kg/injection, in particular 0.8 mCi/kg/injection, i.e. about 0.4 to 40 μmol/kg/injection, in particular 3.2 μmoles/kg/injection. In man, the quantities of labelled derivative injected per protocol represent from 0.25 to 25 mCi/injection, in particular 2.5 mCi/injection, i.e. from 1 to 100 μmol/injection, in particular 10 μmol/injection. These doses correspond to an activity of 2.5 mCi for about 10 μmol of product injected.

By “process for the determination of insulin resistance” is meant a method making it possible to substantiate a variation in the sensitivity or reactivity of the organs to insulin in the course of metabolic processes, in particular mechanisms for storage, circulation and glucose exchange in the organism.

By “variation in the quantity” is meant the variation in the quantity of glucose derivative halogenated in the 6 position, in the cells observed, relative to the total quantity of the aforesaid derivative administered during the administrations which mark the start of the measurements.

By “muscle cells” is meant any cell of the contractile tissue, which includes the striated muscles, smooth muscles and the myocardium.

By “given period Δt” is meant an interval of time, in particular measured in minutes, sufficiently long for the quantity of glucose derivative halogenated in the 6 position no longer to vary significantly in the cells towards which the glucose transport is observed. This time interval lies between 1 and 120 minutes, in particular between 1 and 60 minutes, preferably between 1 and 20 minutes.

By “administration” is meant any form of administration suitable for insulin and for the glucose derivative halogenated in the 6 position, in particular parenteral or oral administration.

By “imaging” is meant a technique which makes it possible to create images from physical data. In the context of the present invention, this is nuclear imaging. Nuclear imaging makes it possible to obtain medical information about a patient or a live animal without recourse to surgery, and consequently avoids the need to work on biological samples.

The purpose of nuclear imaging is to create an intelligible visual representation of information of a medical nature. Nuclear imaging is encountered more generally in the context of medical imaging: the aim is in fact to be able to represent in a relatively simple format a large quantity of information derived from a multitude of measurements acquired according to a well-defined method.

Medical imaging makes it possible to examine a patient without operating on him, that is to say no surgical manoeuvre is performed on the patient, or the animal, during the examination by imaging.

The part of the imaging equipment responsible for collecting the physical data relating to the patient or the animal observed can be placed in contact with the skin or at a distance from the skin of the patient or animal.

According to an advantageous embodiment of the invention, the muscle cells are selected from skeletal muscle cells, cardiac cells or skeletal muscle cells and cardiac cells.

The muscle cells are selected from the cells of the skeletal muscle, which is an organ of particular interest to diabetologists during studies of insulin resistance linked to diabetes. Cardiac cells as this is an organ of particular interest to cardiologists during studies of insulin resistance linked to cardiovascular problems. Skeletal muscle cells and cardiac cells are used, which makes it possible to provide information to both medical field simultaneously.

By “skeletal muscle” is meant the totality of the striated muscles with the exception of the cardiac muscle.

By “cardiac cells” is meant the cells constituting the cardiac muscle.

According to another embodiment of the invention, the muscle cells are rat cells selected from:

skeletal muscle cells,

cardiac cells or

skeletal muscle cells and cardiac cells.

According to another advantageous embodiment of the invention, the muscle cells are human cells selected from:

skeletal muscle cells,

cardiac cells or

skeletal muscle cells and cardiac cells,

and in particular

skeletal muscle cells or

skeletal muscle cells and cardiac cells.

According to an advantageous embodiment of the invention, the glucose derivative halogenated in the 6 position is a pure tracer of glucose transport.

In a particular embodiment of the present invention, the glucose derivative halogenated in the 6 position is a 6-deoxy-6-halogeno-glucose, in particular iodinated or fluorinated, and more particularly 6-deoxy-6-iodoglucose and 6-deoxy-6-fluoroglucose.

The glucose derivatives correspond to the following formula:

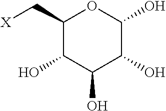

in which X is a halogen atom, in the case of a 6-deoxy-6-halogeno-glucose, X is an iodine atom, in the case of 6-deoxy-6-iodoglucose and X is a fluorine atom, in the case of 6-deoxy-6-fluoroglucose.

By “pure tracer of glucose transport” is meant a molecule making it possible to observe the glucose transport without being influenced by other phenomena, in particular phosphorylation.

By “glucose transport” is meant the totality of the processes of glucose transport across membranes, whether this be via a transporter (for example the transporters GLUT 1-GLUT 4) or by passive diffusion of the glucose.

Certain tracers of glucose transport can be phosphorylated, in particular at the 6 position, and for example 2-deoxy-2-fluoro D-glucose can be mentioned. In case of phosphorylation the molecule of tracer can no longer cross the membrane and remains in the cell. Only the free tracer will be able to cross the membrane again. This type of tracer makes it possible to observe glucose transport and glucose phosphorylation simultaneously.

In the context of the present invention, only the glucose transport is involved. The present invention is concerned with a glucose concentration equilibrium becoming established rapidly between the cells observed and their environment. It is thus necessary to utilise a tracer which cannot be phosphorylated.

Thus a tracer capable of being phosphorylated does not make it possible to implement the present invention.

By “6-deoxy-6-halogeno-glucose” is meant a molecule of glucose, in particular D-glucose, in which the hydroxyl group in the 6 position is replaced by a halogen atom, in particular iodine or fluorine,

According to a particular embodiment of the invention, the glucose derivative halogenated in the 6 position is iodinated, the derivative being in particular 6-deoxy-6-iodoglucose labelled with a radioactive isotope of iodine, in particular iodine 123.

The glucose derivative halogenated in the 6 position bears a halogen atom at the 6 position in place of the hydroxyl group, and is labelled with a radioactive isotope of iodine, in particular iodine 123, 124, 125, 131 or 132, more particularly iodine 123.

By “radioactive isotope” is meant an unstable atom which emits radiation, which may be α, β+, β− or γ, in order to transform itself into another isotope of the same element, or into a different element, more stable than the starting isotope.

By “γ radiation” (gamma) is meant radiation produced by nuclear transitions emitting a very energetic, and thus very penetrating, photon. This radiation is a form of high energy electromagnetic radiation produced by γ disintegrations or other nuclear or subatomic processes. This radiation is detected in the form of number of counts by means for detection of the aforesaid radiation.

By “number of counts” is meant the number of photons emitted during the γ radiation, detected per unit time, the quantity of the aforesaid halogenated derivative as a function of time being determined by the number of counts, resulting from the γ radiation from iodine or from the radioactive fluorine following positron-electron annihilation, detected as a function of time.

By “means for detection” is meant any type of device sufficiently sensitive to detect the emission of a photon emitted via γ radiation, in particular gamma cameras and NaI probes and PET (Positron Emission Tomography) cameras.

The positron β+ from the fluorine, on contact with the electrons of matter, emits two gamma photons at 180° to one another, and it is essential to detect both photons originating from the same positron at the same time in order to be capable of locating the point of collision with the electron (point of annihilation). This type of detection of both the γ's from the positron is called coincidence detection and necessitates the use of special cameras (PET cameras).

According to an advantageous embodiment of the invention, the human cells are selected from

skeletal muscle cells,

cardiac cells or

skeletal muscle cells and cardiac cells,

and in particular

skeletal muscle cells or

skeletal muscle cells and cardiac cells.

According to another embodiment, the present invention relates to a process for the determination of insulin resistance, by means of γ radiation detection, in a mammal capable of exhibiting insulin resistance, in particular a patient, comprising:
a first stage of measurement of the variation in the quantity (as a function of time) of a glucose derivative halogenated in the 6 position, previously administered to the mammal, in particular to the patient, which measurement takes place in muscle cells and optionally the blood of the said mammal, in particular of the said patient, during a given period Δt, by means for detection of γ radiation, to establish a first group of data;
a second stage of measurement of the variation in the quantity (as a function of time) of the aforesaid glucose derivative halogenated in the 6 position, previously administered, following an administration of insulin, to the mammal, in particular to the patient, which measurement takes place in muscle cells and optionally the blood of the said mammal, in particular of the said patient, during a period essentially equal to the aforesaid period Δt, by means for detection of γ radiation, to establish a second group of data;
a third stage of calculation of an index characterizing the rate of glucose transport, the said glucose transport taking place from the blood or the interstitial compartment towards the muscle cells, and the said index being capable of being determined by means of a mathematical algorithm and/or an empirical descriptor, from the aforesaid two groups of data;
a fourth stage of comparison of the aforesaid index characterizing the rate of glucose transport with the index characterizing the rate of glucose transport obtained in a healthy mammal, in particular a healthy patient, by implementing in the said healthy mammal, in particular healthy patient, the three stages defined above with regard to the mammal, in particular the patient, the said comparison making it possible to determine a deviation which can be associated with insulin resistance in the said mammal, in particular in the said patient.
The mathematical models necessitate variations in the quantity of the aforesaid glucose derivative halogenated in the 6 position, in the blood, in order to determine the kinetics which will give the index. The empirical descriptor makes it possible to determine the said index without recourse to kinetic calculations, that is to say to mathematical algorithms. The empirical descriptor makes it possible to dispense with measurement in the blood. The empirical descriptor makes it possible to obtain information directly on the insulin resistance of the cells of the muscle observed.

By “glucose transport” is meant the totality of the processes of glucose transport across membranes, whether this be via a transporter (for example the transporters GLUT 1-GLUT 4) or by passive diffusion of the glucose.

By “interstitial compartment” is meant the environment constituted by the interstitial liquid which fills the space between the blood vessels and the cell; this liquid facilitates the exchanges of nutrients and wastes between the blood vessels and the cells.

By “mathematical algorithm” is meant an equation deriving from the mathematical modelling of physiological phenomena.

By “empirical descriptor” is meant an equation arbitrarily established, but selected since it makes it possible to obtain results close to those obtained by the mathematical modelling of physiological phenomena.

By “determined by means of a mathematical algorithm and/or an empirical descriptor” is meant the mathematical calculation of the index.

By groups of data is meant a set of values obtained during a series of measurements relating to a common subject.

By “healthy mammal” and “healthy patient” is meant a mammal and a patient which do not exhibit any pathology, metabolic abnormality or other physiological disorder, and more precisely do not exhibit any form of insulin resistance.

By “comparison” is meant the relating of two values to determine whether they comprise a significant deviation after allowance for measurement uncertainties.

By “deviation which can be associated with insulin resistance” is meant a significant difference between the values of the index characterizing the rate of glucose transport in the patient observed and the healthy patient, and the possibility that this deviation in value signifies a disorder of the glucose metabolism connected with insulin resistance.

According to an advantageous embodiment, the invention relates to a process in which the index calculated from a mathematical algorithm is the theoretical index, the said theoretical index corresponding in particular to the ratio of the kinetics of glucose transport taking place from the blood or the interstitial compartment towards the muscle cells.

The mathematical algorithm is an equation making it possible to calculate the theoretical index which is a value; the mathematical algorithms utilised in the present invention enable the calculation of the kinetics of glucose transport; for this, it is necessary to know the variations in quantity of glucose in the different cellular (muscle) and extracellular (blood) compartments.

According to another embodiment of the invention, the process for the determination of insulin resistance is carried out in a patient capable of exhibiting insulin resistance, in which the muscle cells are skeletal muscle cells, and comprising a third stage of calculation of an index characterizing the rate of glucose transport, the said glucose transport taking place from the interstitial compartment towards the skeletal muscle cells, and the said index being obtained by a mathematical algorithm or an empirical descriptor from the aforesaid two groups of data.

In the case of the skeletal muscle, the glucose transport does not take place directly from the blood to muscle cells, there is an intermediate compartment called the interstitial compartment. The mathematical model has been adapted to take account of this compartment, since the variation in concentration of glucose tracer in the interstitial compartment cannot be measured directly as is the case in the blood. Thus, the algorithm deriving from this mathematical model makes it possible to calculate the kinetics of glucose transport from the interstitial compartment towards the skeletal muscle cells by using the data indicating the variations in quantities of glucose tracer in the blood and the skeletal muscle. In the context of the invention, we are concerned with the kinetics of glucose transport from the interstitial compartment towards the skeletal muscle cells, that is to say the entry of the glucose into the skeletal muscle cells, since this stage is stimulated by insulin.

The possibility of easily measuring the muscular insulin resistance will enable the diabetologist to establish a precise diagnosis, and thus to adapt the therapy and to ensure its monitoring in terms of efficacy. Moreover, it will be possible for a diabetologist to obtain pertinent information, by means of a NaI probe, in 45 minutes and at the consultation site, to determine whether or not a patient exhibits muscular insulin resistance.

According to another embodiment of the invention, the process for the determination of insulin resistance is implemented in a patient capable of exhibiting insulin resistance, in which the muscle cells are cardiac cells, and comprising a third state of calculation of an index characterizing the rate of glucose transport, the said glucose transport taking place from the blood towards the cardiac cells, and the said kinetics being obtained by a mathematical algorithm or an empirical descriptor from the aforesaid two groups of data;

In the case of the heart, the glucose transport takes place directly from the blood towards the cells of the cardiac muscle; the mathematical model utilised makes it possible to obtain the kinetics of glucose transport from the blood towards the cardiac cells, from data relating to the variations in quantity of glucose tracer in the blood and in the cardiac cells. The algorithm deriving from the mathematical model thus makes it possible to obtain the kinetics of glucose transport from the blood towards the cardiac cells, that is to say on the entry of the glucose into the cardiac cells. In the context of the invention we are concerned with these entry kinetics since this stage is stimulated by insulin.

It is now known that insulin resistance is a fully-fledged risk factor for cardiovascular diseases. Measurement of the cardiac insulin resistance can aid the practitioner in diagnosing a cardiac insulin resistance and enable the management of the patients at risk, with immuno-sensitisers for example. This help to diagnosis also enables better monitoring of the therapy and also personalisation of the treatment.

According to a particular embodiment, the invention describes a process for the determination of insulin resistance in a patient capable of exhibiting insulin resistance, in which the muscle cells are skeletal muscle cells and cardiac cells, and comprising
a first stage comprising
   a measurement of the variation in the quantity (as a function of time) of a glucose derivative halogenated in the 6 position, previously administered to the patient, which measurement takes place in skeletal muscle cells of the said patient, to establish a first group of data relating to the skeletal muscle, and
   a measurement of the variation in the quantity (as a function of time) of a glucose derivative halogenated in the 6 position, previously administered to the patient, which measurement takes place in cardiac cells of the said patient, to establish a first group of data relating to the heart,
a second stage comprising
   a measurement of the variation in the quantity (as a function of time) of a glucose derivative halogenated in the 6 position, previously administered after an administration of insulin, to the patient, which measurement takes place in skeletal muscle cells of the said patient, to establish a second group of data relating to the skeletal muscle, and
   a measurement of the variation in the quantity (as a function of time) of a glucose derivative halogenated in the 6 position, previously administered after an administration of insulin, to the patient, which measurement takes place in cardiac cells of the said patient, to establish a second group of data relating to the heart,
a third stage of calculation comprising
   the calculation of an index characterizing the rate of glucose transport, the said glucose transport taking place from the interstitial compartment towards the skeletal muscle cells, and the said index being capable of being determined by means of a mathematical algorithm and/or an empirical descriptor from the aforesaid two groups of data relating to the skeletal muscle, and
   the calculation of an index characterizing the rate of glucose transport, the said glucose transport taking place from the blood towards the cardiac cells, and the said index being capable of being determined by means of a mathematical algorithm and/or an empirical descriptor from the aforesaid two groups of data relating to the heart;
a fourth stage of comparison
   of the aforesaid index characterizing the rate of glucose transport, into the skeletal muscle cells, with the index characterizing the rate of glucose transport, into the skeletal muscle cells, obtained in a healthy patient, by implementing in the said healthy patient the three stages as defined above, the deviation making it possible to characterize the insulin resistance of the said patient.
   of the aforesaid index characterizing the rate of glucose transport, into the cardiac cells, with the index characterizing the rate of glucose transport, into the cardiac cells, obtained in a healthy patient, by implementing in the said healthy patient the three stages as defined above, the deviation making it possible to characterize the insulin resistance of the said patient.

The advantage of measuring simultaneously on both organs is to enable greater reliability in the determination of insulin resistance, and to provide information on two organs which are insulin sensitive but can have different responses to insulin, while ensuring that these data were collected at the same moment and that therefore the external parameters are the same for both measurements, to complement a clinical picture enabling the diagnosis of diseases connected with insulin resistance.

The only data utilised by the clinician are the patient's glycaemia and insulinemia values, which give information on a general metabolic disequilibrium and on a possible general insulin resistance. The possibility of measuring the resistance of each organ to insulin opens up a new field of investigation in physiopathology since nothing is known of the chronology of the appearance of insulin resistance in the different organs. This information should enable better management of the patient with a possibly more pertinent therapeutic approach and, above all, monitoring of the efficacy of the treatment.

In the context of the invention it is also possible to measure the variations in quantities of glucose tracer in the blood in order to constitute two groups of data relating to the blood. This measurement can be carried out by taking blood samples, then measuring the γ radiation from these samples to determine the quantity of tracer which they contain; or by direct measurement of the γ radiation over a region of the body of the mammal, or the patient, pertinent for this measurement, such as the aortic arch. This latter method has the advantage of not requiring blood sampling, hence it is much less restricting for the patient.

The measurements of quantities of tracer in the blood make it possible to determine the glucose transport index by means of the mathematical algorithm.

According to another embodiment, the invention describes a process for the determination of insulin resistance in a mammal capable of exhibiting insulin resistance, in particular a patient, comprising
a first stage, performed during a given period Δt, comprising
   a measurement, by means for detection of γ radiation, of the variation in the quantity (as a function of time) of a glucose derivative halogenated in the 6 position, previously administered to the mammal, in particular to the patient, which measurement takes place on blood samples from the said mammal, in particular from the said patient, the said samples having been taken during the aforesaid given period Δt, to establish a first group of data relating to the blood, and
   a measurement, by means for detection of γ radiation, of the variation in the quantity (as a function of time) of a glucose derivative halogenated in the 6 position, previously administered to the mammal, in particular to the patient, which measurement takes place in muscle cells of the said mammal, in particular of the said patient, to establish a first group of data relating to the muscle;
a second stage, performed during a period essentially equal to the aforesaid period Δt, comprising
   a measurement, by means for detection of γ radiation, of the variation in the quantity (as a function of time) of a glucose derivative halogenated in the 6 position, previously administered after an administration of insulin, to the mammal, in particular to the patient, which measurement takes place on blood samples from the said mammal, in particular from the said patient, the said samples having been taken during the aforesaid given period Δt, to establish a second group of data relating to the blood, and
   a measurement, by means for detection of γ radiation, of the variation in the quantity (as a function of time) of a glucose derivative halogenated in the 6 position, previously administered after an administration of insulin, to the mammal, in particular to the patient, which measurement takes place in muscle cells of the said mammal, in particular of the said patient, to establish a second group of data relating to the muscle;
a third stage of calculation of an index characterizing the rate of glucose transport, the said glucose transport taking place from the blood towards the muscle cells, and the said index being determined by means of a mathematical algorithm, the calculation of this index making use of the groups of data relating to the blood, and the groups of data relating to the muscle,
a fourth stage of comparison of the aforesaid index characterizing the rate of glucose transport with the index characterizing the rate of glucose transport obtained in a healthy mammal, in particular a healthy patient, by implementing in the said healthy mammal, in particular healthy patient, the three stages defined above with regard to the mammal, in particular the patient, the said comparison making it possible to determine a deviation which can be associated with insulin resistance in the said mammal, in particular in the said patient.

By “blood samples” is meant volumes of blood sampled from the mammal, in particular from the patient, at defined intervals, intravenously or by means of a catheter.

According to another embodiment, the invention describes a process for the determination of insulin resistance in a mammal capable of exhibiting insulin resistance, in particular a patient, comprising:
a first stage, performed during a given period Δt, comprising
   a measurement, by means for detection of γ radiation, of the variation in the quantity (as a function of time) of a glucose derivative halogenated in the 6 position, previously administered to the mammal, in particular to the patient, which measurement takes place in muscle cells of the said mammal, in particular of the said patient, to establish a first group of data relating to the muscle, and
   a measurement, by means for detection of γ radiation, of the variation in the quantity (as a function of time) of a glucose derivative halogenated in the 6 position, previously administered to the mammal, in particular to the patient, which measurement takes place in the blood of the said mammal, in particular of the said patient, to establish a first group of data relating to the blood,
a second stage, performed during a period essentially equal to the aforesaid period Δt, comprising
   a measurement, by means for detection of γ radiation, of the variation in the quantity (as a function of time) of a glucose derivative halogenated in the 6 position, previously administered after an administration of insulin, to the mammal, in particular to the patient, which measurement takes place in muscle cells of the said mammal, in particular of the said patient, to establish a second group of data relating to the muscle, and
   a measurement, by means for detection of γ radiation, of the variation in the quantity (as a function of time) of a glucose derivative halogenated in the 6 position, previously administered after an administration of insulin, to the mammal, in particular to the patient, which measurement takes place in the blood of the said mammal, in particular of the said patient, to establish a second group of data relating to the blood,
a third stage of calculation of an index characterizing the rate of glucose transport, the said glucose transport taking place from the blood towards the muscle cells, and the said index being determined by means of a mathematical algorithm, the calculation of this index making use of the groups of data relating to the blood, and the groups of data relating to the muscle,
a fourth stage of comparison of the aforesaid index characterizing the rate of glucose transport with the index characterizing the rate of glucose transport obtained in a healthy mammal, in particular a healthy patient, by implementing in the said healthy mammal, in particular healthy patient, the three stages defined above with regard to the mammal, in particular the patient, the said comparison making it possible to determine a deviation which can be associated with insulin resistance in the said mammal, in particular in the said patient.

These measurements of the variation in the quantity of glucose tracer in the blood are necessary to make it possible to calculate the theoretical glucose transport index.

These measurements make it possible simultaneously to obtain the variations in quantity of glucose tracer in the blood and in the muscle cells, under conditions referred to as “basal” and “insulin”. From these values it is possible to calculate the glucose transport index by using a mathematical model suitable for the type of muscle cells considered.

Advantageously, the process for measurement of the variation in the quantity of glucose tracer in the blood, using a means for detection of γ radiation, without needing to take blood samples, makes it possible to reduce the clinical constraints on the mammal, in particular the patient, on which it is desired to detect insulin resistance.

According to another advantageous embodiment, the invention relates to a process in which the index calculated from an empirical descriptor is the empirical index, the said empirical descriptor itself being obtained from the aforesaid groups of data, by one or more mathematical operations in particular:
additions, subtractions, multiplications and divisions on the totality, a part or on parts of each of the two groups of data; and/or,
integrations and derivations on graphic representations, in particular curves, obtained from the totality, a part or on parts of each of the two groups of data.

The empirical descriptor is an equation, determined empirically, making it possible to calculate the empirical index which is a value.

According to another embodiment the invention relates to a process comprising the following stages making it possible to select an empirical descriptor:
1—determination of the theoretical index by means of a mathematical algorithm, the said theoretical index then corresponding in particular to the ratio of the kinetics of glucose transport,
2—determination of a group of empirical descriptors making it possible to obtain a group of empirical indices, each of the said empirical descriptors being obtained from the aforesaid two groups of data, by one or more mathematical operations;
3—comparison of the empirical indices with the theoretical index, in order to determine the empirical index closest to the theoretical index, and to select the empirical descriptor corresponding to that empirical index.

The empirical descriptor is an equation arbitrarily selected for its ability to provide the closest possible indices to the indices obtained with the mathematical algorithm, which is also an equation, when the same sets of data are processed with these two equations.

By way of example, the following empirical descriptor was determined by means of the process described above:

Activity [(10 min insulin×20 min insulin)/(10 min basal×20 min basal)]×[Ratio (50-90)×Ratio (900-1200)]

The meaning of the terms used in this equation is given in detail in the experimental part of the invention.

By “empirical index closest to the theoretical index” is meant the value of the empirical index having a difference from the value of the theoretical index of the lowest possible significance. The lowest significance limits utilised are p≦0.05, with for correlation r²≧0.45, in particular r²≧0.45 and less than 0.75, preferably r²≧0.75.

According to another embodiment of the invention, the mammal is the rat and the cells are skeletal muscle cells or skeletal muscle cells and cardiac cells.

According to an advantageous embodiment of the invention, the glucose derivative halogenated in the 6 position is a pure tracer of glucose transport.

In a particular embodiment of the present invention, the glucose derivative halogenated in the 6 position is a 6-deoxy-6-halogeno-glucose, in particular iodinated or fluorinated, and more particularly 6-deoxy-6-iodoglucose and 6-deoxy-6-fluoroglucose.

According to a particular embodiment of the invention, the glucose derivative halogenated in the 6 position is iodinated, the derivative being in particular 6-deoxy-6-iodoglucose labelled with a radioactive isotope of iodine, in particular iodine 123.

The glucose derivative halogenated in the 6 position is in particular 6-deoxy-6-iodoglucose labelled with a radioactive isotope of iodine, in particular iodine 123, 124, 125, 131 or 132, and more particularly iodine 123.

According to another embodiment, the invention relates to a process for the determination of insulin resistance, in a patient capable of exhibiting insulin resistance, in which the stages of measurement of the variation in the quantity of the aforesaid glucose derivative halogenated in the 6 position in skeletal muscle cells are carried out by means of a probe enabling the detection of γ radiation from iodine and γ radiation from the positron-electron annihilation of fluorine, in particular a NaI probe.

According to another embodiment, the invention relates to a process for the determination of insulin resistance, in a patient capable of exhibiting insulin resistance, in which the stages of measurement of the variation in the quantity of the aforesaid glucose derivative halogenated in the 6 position in cardiac cells are carried out using a means for detection of γ radiation from iodine and γ radiation from the positron-electron annihilation of fluorine, in particular a γ camera or a PET camera.

According to another embodiment, the invention relates to a process for the determination of insulin resistance, in a patient capable of exhibiting insulin resistance, in which the stages of measurement of the variation in the quantity of the aforesaid glucose derivative halogenated in the 6 position in skeletal muscle cells and cardiac cells are carried out by means of a probe enabling the detection of γ radiation from iodine and from fluorine, in particular a NaI probe for the skeletal muscle cells, and a means for detection of γ radiation from iodine and γ radiation from the positron-electron annihilation of fluorine, in particular a γ camera or a PET camera for the cardiac cells.

According to another embodiment, the invention relates to a process for the determination of insulin resistance, in a patient capable of exhibiting insulin resistance, in which the stages of measurement of the variation in the quantity of the aforesaid glucose derivative halogenated in the 6 position in the blood are carried out using a means for detection of γ radiation from iodine and from fluorine, in particular a γ camera, a PET camera or a γ counter.

The measurements of the quantity of tracer carried out on the blood samples are performed by means of the γ counter, and the measurements in the quantity of tracer carried out in proximity to the surface of the patient's skin are performed by means of a γ camera or a PET camera. These measurements make it possible to determine the theoretical glucose transport index, by means of mathematical algorithms, the said theoretical index making it possible to determine an empirical descriptor.

Thus, the measurement on the blood is necessary in order to be able to determine an empirical descriptor. However, once the empirical descriptor is known it is no longer necessary to measure the variations in quantity of tracer in the blood, since the insulin resistance can be determined with the groups of data relating to the muscle in question and the empirical descriptor.

According to a particular embodiment, the invention relates to a process for the determination of insulin resistance in a patient, comprising
a first stage of measurement of the variation in the quantity (as a function of time) of 6-deoxy-6-iodoglucose previously injected into the patient, which measurement takes place in skeletal muscle cells of the said patient, during a given period Δt, in particular about 20 minutes from the injection of 6-deoxy-6-iodoglucose, which measurement takes place by means of a probe in particular NaI enabling the detection of γ radiation from iodine 123, to establish a first group of data;
a second stage of measurement of the variation in the quantity (as a function of time) of the 6-deoxy-6-iodoglucose previously injected, and preceded by an injection of insulin, in particular about 10 minutes before the injection of the aforesaid iodinated derivative into the patient, which measurement takes place in skeletal muscle cells of the said patient, during a given period Δt, in particular about 20 minutes, from the injection of 6-deoxy-6-iodoglucose, which measurement takes place by means of a probe in particular NaI enabling the detection of γ radiation from iodine 123, to establish a second group of data;
a third stage of calculation of an index characterizing the rate of glucose transport, the said glucose transport taking place from the interstitial compartment towards the skeletal muscle cells, and the said index being capable of being determined by means of a mathematical algorithm and/or an empirical descriptor, from the aforesaid two groups of data;
a fourth stage of comparison of the aforesaid index characterizing the rate of glucose transport with the index characterizing the rate of glucose transport obtained in a healthy patient, by implementing in the said healthy patient the three stages defined above with regard to the patient, the said comparison making it possible to determine a deviation which can be associated with insulin resistance of the said patient.

According to a particular embodiment, the invention relates to a process for the determination of insulin resistance in a patient, comprising
a first stage of measurement of the variation in the quantity (as a function of time) of 6-deoxy-6-iodoglucose previously injected into the patient, which measurement takes place in cardiac cells of the said patient, during a given period Δt, in particular about 20 minutes, from the injection of 6-deoxy-6-iodoglucose, which measurement takes place by means in particular of a γ camera enabling the detection of γ radiation from iodine 123, to establish a first group of data;
a second stage of measurement of the variation in the quantity (as a function of time) of the 6-deoxy-6-iodoglucose previously injected, and preceded by an injection of insulin, in particular about 10 minutes before the injection of the aforesaid iodinated derivative into the patient, which measurement takes place in cardiac cells of the said patient, during a given period Δt, in particular about 20 minutes, which measurement takes place by means in particular of a γ camera enabling the detection of γ radiation from iodine 123, to establish a second group of data;
a third stage of calculation of an index characterizing the rate of glucose transport, the said glucose transport taking place from the blood towards the cardiac cells, and the said index being capable of being determined by means of a mathematical algorithm and/or an empirical descriptor, from the aforesaid two groups of data;
a fourth stage of comparison of the aforesaid index characterizing the rate of glucose transport with the index characterizing the rate of glucose transport obtained in a healthy patient, by implementing in the said healthy patient the three stages defined above with regard to the patient, the said comparison making it possible to determine a deviation which can be associated with insulin resistance of the said patient.

According to a particular embodiment, the invention relates to a process for the determination of insulin resistance in a patient, comprising
a first stage comprising
   a measurement of the variation in the quantity (as a function of time) of 6-deoxy-6-iodoglucose previously injected into the patient, which measurement takes place in skeletal muscle cells of the said patient, during a given period Δt, in particular about 20 minutes, from the injection of 6-deoxy-6-iodoglucose, which measurement takes place by means of a probe in particular NaI enabling the detection of γ radiation from iodine 123, to establish a first group of data relating to the skeletal muscle, and;
   a measurement of the variation in the quantity (as a function of time) of 6-deoxy-6-iodoglucose previously injected into the patient, which measurement takes place in cardiac cells of the said patient, during a given period Δt, in particular about 20 minutes, from the injection of 6-deoxy-6-iodoglucose, which measurement takes place by means in particular of a γ camera enabling the detection of γ radiation from iodine 123, to establish a first group of data relating to the heart;
a second stage comprising
   a measurement of the variation in the quantity (as a function of time) of the 6-deoxy-6-iodoglucose previously injected, and preceded by an injection of insulin, in particular about 10 minutes before the injection of the aforesaid iodinated derivative into the patient, which measurement takes place in skeletal muscle cells of the said patient, during a given period Δt, in particular about 20 minutes, from the injection of 6-deoxy-6-iodoglucose, which measurement takes place by means of a probe in particular NaI enabling the detection of γ radiation from iodine 123, to establish a second group of data relating to the skeletal muscle, and;
   a measurement of the variation in the quantity (as a function of time) of the 6-deoxy-6-iodoglucose previously injected, and preceded by an injection of insulin, in particular about 10 minutes before the injection of the aforesaid iodinated derivative into the patient, which measurement takes place in cardiac cells of the said patient, during a given period Δt, in particular about 20 minutes, from the injection of 6-deoxy-6-iodoglucose, which measurement takes place by means in particular of a γ camera enabling the detection of y radiation from iodine 123, to establish a second group of data relating to the heart,
a third stage of calculation comprising
   the calculation of an index characterizing the rate of glucose transport, the said glucose transport taking place from the interstitial compartment towards the skeletal muscle cells, and the said index being capable of being determined by means of a mathematical algorithm and/or an empirical descriptor, from the aforesaid two groups of data relating to the skeletal muscle, and;
   the calculation of an index characterizing the rate of glucose transport, the said glucose transport taking place from the blood towards the cardiac cells, and the said index being capable of being determined by means of a mathematical algorithm and/or an empirical descriptor, from the aforesaid two groups of data relating to the heart;
a fourth stage of comparison
   of the aforesaid index characterizing the rate of glucose transport, into the skeletal muscle cells, with the index characterizing the rate of glucose transport, into the skeletal muscle cells obtained in a healthy patient, by implementing in the said healthy patient the three stages defined above with regard to the patient, the said comparison making it possible to determine a deviation which can be associated with insulin resistance of the said patient, and;
   of the aforesaid index characterizing the rate of glucose transport, into the cardiac cells, with the index characterizing the rate of glucose transport, into the cardiac cells, obtained in a healthy patient, by implementing in the said healthy patient the three stages defined above with regard to the patient, the said comparison making it possible to determine a deviation which can be associated with insulin resistance of the said patient.

In the context of the invention it is also possible to measure the variations in quantities of glucose tracer in the patient's blood, in order to constitute two groups of data relating to the blood. This measurement can be carried out by taking blood samples, then measuring the y radiation from these samples by means of a γ counter to determine the quantity of tracer which they contain; or by direct measurement of the γ radiation over a region of the patient's body, by means of a γ camera, this region having to be pertinent for this measurement, such as the aortic arch. This latter method has the advantage of not requiring blood sampling, hence it is much less restrictive for the patient.

The measurements of quantities of tracer in the blood make it possible to determine the glucose transport index by means of the mathematical algorithm.

According to a particular embodiment, the invention relates to a process for the determination of insulin resistance in a patient, comprising
a first stage, performed during a given period Δt, comprising
   a measurement of the variation in the quantity (as a function of time) of 6-deoxy-6-iodoglucose previously injected into the patient, which measurement takes place on blood samples from the said patient, the said samples having been taken during the aforesaid given period Δt, in particular about 20 minutes, from the injection of 6-deoxy-6-iodoglucose, which measurement takes place by means of a gamma counter, enabling the detection of the γ radiation from iodine 123, to establish a first group of data relating to the blood, and
   a measurement of the variation in the quantity (as a function of time) of 6-deoxy-6-iodoglucose previously injected into the patient, which measurement takes place in muscle cells of the said patient, during the aforesaid given period Δt, in particular about 20 minutes, from the injection of 6-deoxy-6-iodoglucose, which measurement takes place by means of a γ camera, or a Nat probe, enabling the detection of the γ radiation from iodine 123, to establish a first group of data relating to the muscle,
a second stage, performed during a period essentially equal to the aforesaid period Δt, comprising
   a measurement of the variation in the quantity (as a function of time) of 6-deoxy-6-iodoglucose previously injected, and preceded by an injection of insulin, in particular about 10 minutes before the injection of the aforesaid iodinated derivative into the patient, which measurement takes place on blood samples from the said patient, the said samples having been taken during the aforesaid given period Δt, in particular about 20 minutes, from the injection of 6-deoxy-6-iodoglucose, which measurement takes place by means of a gamma counter, enabling the detection of the γ radiation from iodine 123, to establish a second group of data relating to the blood, and
   a measurement of the variation in the quantity (as a function of time) of 6-deoxy-6-iodoglucose previously injected, and preceded by an injection of insulin, in particular about 10 minutes before the injection of the aforesaid iodinated derivative into the patient, which measurement takes place in muscle cells of the said patient, during the aforesaid given period Δt, in particular about 20 minutes, from the injection of 6-deoxy-6-iodoglucose, which measurement takes place by means of a γ camera or a NaI probe, enabling the detection of the γ radiation from iodine 123, to establish a second group of data relating to the muscle,
a third stage of calculation of an index characterizing the rate of glucose transport, the said glucose transport taking place from the blood towards the muscle cells, and the said index being determined by means of a mathematical algorithm, the calculation of this index making use of the groups of data relating to the blood, and the groups of data relating to the muscle,
a fourth stage of comparison of the aforesaid index characterizing the rate of glucose transport with the index characterizing the rate of glucose transport obtained in a healthy patient, by implementing in the said healthy patient the three stages defined above with regard to the patient, the said comparison making it possible to determine a deviation which can be associated with insulin resistance of the said patient.

According to a particular embodiment, the invention relates to a process for the determination of insulin resistance in a patient, comprising
a first stage, performed during a given period Δt, comprising
   a measurement of the variation in the quantity (as a function of time) of 6-deoxy-6-iodoglucose previously injected into the patient, which measurement takes place in the blood of the said patient, during the aforesaid given period Δt, in particular about 20 minutes, from the injection of 6-deoxy-6-iodoglucose, which measurement takes place by means of a γ camera, enabling the detection of the γ radiation from iodine 123, to establish a first group of data relating to the blood, and
   a measurement of the variation in the quantity (as a function of time) of 6-deoxy-6-iodoglucose previously injected into the patient, which measurement takes place in muscle cells of the said patient, during the aforesaid given period Δt, in particular about 20 minutes, from the injection of 6-deoxy-6-iodoglucose, which measurement takes place by means of a γ camera, or a NaI probe, enabling the detection of the γ radiation from iodine 123, to establish a first group of data relating to the muscle,
a second stage, performed during a period essentially equal to the aforesaid period Δt, comprising
   a measurement of the variation in the quantity (as a function of time) of 6-deoxy-6-iodoglucose previously injected, and preceded by an injection of insulin, in particular about 10 minutes before the injection of the aforesaid iodinated derivative into the patient, which measurement takes place in the blood of the said patient, during the aforesaid given period Δt, in particular about 20 minutes, from the injection of 6-deoxy-6-iodoglucose, which measurement takes place by means of a γ camera, enabling the detection of the γ radiation from iodine 123, to establish a second group of data relating to the blood, and
   a measurement of the variation in the quantity (as a function of time) of 6-deoxy-6-iodoglucose previously injected, and preceded by an injection of insulin, in particular about 10 minutes before the injection of the aforesaid iodinated derivative into the patient, which measurement takes place in muscle cells of the said patient, during the aforesaid given period Δt, in particular about 20 minutes, from the injection of 6-deoxy-6-iodoglucose, which measurement takes place by means of a γ camera, or a NaI probe, enabling the detection of the γ radiation from iodine 123, to establish a second group of data relating to the muscle,
a third stage of calculation of an index characterizing the rate of glucose transport, the said glucose transport taking place from the blood towards the muscle cells, and the said index being determined by means of a mathematical algorithm, the calculation of this index making use of the groups of data relating to the blood, and the groups of data relating to the muscle,
a fourth stage of comparison of the aforesaid index characterizing the rate of glucose transport with the index characterizing the rate of glucose transport obtained in a healthy patient, by implementing in the said healthy patient the three stages defined above with regard to the patient, the said comparison making it possible to determine a deviation which can be associated with insulin resistance of the said patient.

The experimental part describes the experiments performed in the context of the present invention, in particular experiments on the sensitivity and reproducibility of measurements according to the present invention. The sensitivity experiments utilise rosiglitazone to reestablish sensitivity to insulin in diabetic rats.

By “sensitivity” is meant the ability of the tracer (6DIG) to reveal the variations in glucose transport and the variations in insulin resistance, and the ability of the empirical descriptor, the theoretical index and the empirical index according to the present invention, to detect insulin resistance, that is to say a statistically significant abnormality in glucose transport. The descriptor, or the index, exhibits an ability to detect variations in the glucose transport, sufficient to determine the insulin resistance of a mammal.

By “reproducibility [symbol] is meant the ability of the empirical descriptor, the theoretical index and the empirical index according to the present invention to detect insulin resistance in mammals without statistically significant variations in the result. That is to say that the same experiment can be performed several times and produce the same result at each attempt.

By “statistically significant variation” is meant a variation tested according to a Mann and Whitney statistical test. The result is considered significant if p is less than or equal to 0.05.

Rosiglitazone is a antidiabetic intended for the treatment of type 2 diabetes. Rosiglitazone is an agonist of the gamma PPAR (peroxisome proliferator-activated receptor); it reduces the availability of lipids, and improves the action of insulin and glycoregulation. Chemically, rosiglitazone is (±)-5-[[4-[2-(methyl-2-pyridinylamino)ethoxy]phenyl]methyl]-2,4-thiazolidinedione, and its CAS number is 122320-73-4.

### DESCRIPTION OF FIGURES

FIG. 1: Sequence of Injections

FIG. 1 describes a sequence of injections of 6DIG and insulin in order to determine insulin resistance. The figure represents a chronology starting on the left of the figure at time 1 corresponding to the first injection of 6DIG. From this time 1, and over a period of 10 to 30 minutes, in particular 20 minutes, the measurement A corresponding to the acquisition of data under the condition referred to as “basal” takes place. The time 2 directly follows the measurement A; it corresponds to an injection of insulin. 10 minutes after this time 2, there occurs the time 3 corresponding to the second injection of 6DIG. From this time 3, and over a period of 20 minutes, the measurement B corresponding to the acquisition of data under the condition referred to as “insulin” takes place.

FIG. 2.: Model of Compartments in the Case of Skeletal Muscle

FIG. 2 represents the different compartments used in the calculation of the kinetics of glucose exchange with the skeletal muscle cells. In this figure, Cₚ represents the plasma compartment (blood), Cᵢ the interstitial compartment and Cₑ the compartment corresponding to the skeletal muscle cells. The kinetic constants of glucose exchange between the different compartments are designated k1 for the exchange from the blood towards the interstitial medium, k2 for the exchange from the interstitial medium towards the blood, k3 for the exchange from the interstitial medium towards the skeletal muscle cells and k4 for the exchange from the skeletal muscle towards the interstitial medium.

FIG. 3.: Model of Compartments in the Case of the Heart.

FIG. 3 represents the different compartments used in the calculation of the kinetics of glucose transport during the exchanges with the cardiac cells. In this figure, q1 represents the plasma compartment (blood), q2 the compartment corresponding to the cardiac cells and q3 the compartment representing the peripheral tissues.

The kinetic constants of glucose transport between the different compartments are designated k(i,j), i and j being whole numbers associated with the compartments. In this representation, i represents the compartment towards which the transport takes place and j the compartment from which the transport takes place. Thus, k(2,1) represents the kinetics of glucose transport taking place from the blood towards the heart, and k(0.1) represents an irreversible outflow (elimination via the kidneys for example).

FIG. 4.: Correlation Between the Theoretical and Empirical Indices (Rat Skeletal Muscle)

FIG. 4 represents the correlation between the empirical and theoretical glucose transport indices, in the skeletal muscle cells. The values of the theoretical index are shown on the x-axis and the values of the empirical index on the y-axis. Each dot corresponds to one rat, the black dots are Zucker rats (insulin resistant), and the white dots are Wistar rats (healthy). The measurement protocol was repeated for each rat, the theoretical and empirical indices determined for each rat, and then a dot designating the rat is plotted in FIG. 4 as a function of its two indices. The solid straight line represents the regression line of the all of the rats.

FIG. 5.: Significant Deviation by Means of the Empirical Index

FIG. 5 represents the significant separation which can be observed between the healthy rats (Wistar, white dots) and the insulin resistant rats (Zucker, black dots), by means of the 6DIG measurement protocol and by means of an empirical descriptor. The empirical index of glucose transport is shown on the y-axis in the figure. This index was calculated for each of the rats representing a dot on the column situated in the centre of the figure. The data were obtained in the context of a protocol where the results acquisition period is 20 minutes in “basal” condition and 20 minutes in “insulin” condition. The two isolated dots to the right of the column represent the means for the Wistar and Zucker rats with their standard deviations.

FIG. 6.: Mean of the Coefficients k3 for the Rat (Muscle)

This graph represents the mean obtained during the calculations of k3, namely the coefficients of glucose transfer into skeletal muscle cells. The values of k3 are shown on the y-axis. The x-axis comprises 4 columns: the two on the left and the two on the right; the two on the left relate to the values observed for the Wistar rats (healthy), and the two on the right relate to the Zucker rats (insulin resistant). The white columns show the values of k3 in the condition referred to as “basal”, that is to say without injection of insulin; the black columns show the values of k3 in the condition referred to as “insulin”, that is to say after injection of insulin.

FIG. 7.: Variation in the Coefficients k3 for the Rat (Muscle)

This graph represents the variations obtained during the calculations of k3, namely the coefficients of glucose transfer into skeletal muscle cells. The values of k3 are shown on the y-axis. The x-axis indicates whether the values were recorded in the “basal” state that is to say without injection of insulin (left column); or in the “insulin” state that is to say after injection of insulin (right column). The continuous lines link the white dots which mark the values obtained for Wistar rats (healthy), and the dashed lines link the black dots which mark the values obtained for the Zucker rats (insulin resistant).

The isolated white dot to the right of the column representing the measurements in the “insulin” state indicates the mean value measured in the “insulin” state for the Wistar rats and the standard deviation with a discrimination of p=0.003.

The isolated black dot to the right of the column representing the measurements in the “insulin” state indicates the mean value measured in the “insulin” state for the Zucker rats and the standard deviation with a discrimination of p=0.003.

FIG. 8.: Variations in Quantity of 6DIG in the Dog Heart.

This graph represents on the y-axis the variations in bpm (beats per minute), for one pixel and in the quantity (in mCi) of 6DIG injected, in relation to the x-axis which represents the data acquisition time in minutes, in the heart of a dog. The measured activity is related to the number of pixels, since when a region of interest is determined on a scintigraphy image, the number of pixels in the manually selected zone is not always the same.

Two series of data are shown; a first series of measurements indicated by linked squares represents the data recorded for a fasting animal. A second series of data, indicated by linked circles, represents the data recorded for an animal perfused with a GIK solution (Glucose/Insulin/potassium).

FIG. 9.: Correlation Between Theoretical and Empirical Indices (Rat Heart)

FIG. 9 represents the correlation between the empirical and theoretical glucose transport indices, in the cardiac cells, in the rat. The values of the theoretical index are shown on the x-axis, the values of the empirical index on the y-axis. Each dot corresponds to one rat, the black dots are Zucker rats (n=11, insulin resistant) and the white dots are the Wistar rats (n=11, healthy). The measurement protocol was repeated for each rat, the theoretical and empirical indices determined for each rat, and then a dot designating the rat is plotted in FIG. 9 as a function of its two indices. The solid straight line represents the regression line of the all of the rats (R²=0.7253; y=0.7696*x+0.1111).

FIG. 10.: Correlation Between Theoretical and Empirical Indices (Rat Skeletal Muscle).

FIG. 10 represents the correlation between the empirical and theoretical glucose transport indices, in the skeletal muscle cells, in the rat. The values of the theoretical index are shown on the x-axis, the values of the empirical index on the y-axis. Each dot corresponds to one rat, the black dots are Zucker rats (n=9, insulin resistant) and the white dots are the Wistar rats (n=10, healthy). The measurement protocol was repeated for each rat, the theoretical and empirical indices determined for each rat, and then a dot designating the rat is plotted in FIG. 10 as a function of its two indices. The solid straight line represents the regression line of the all of the rats (R²=0.6344; y=0.4225*x+0.6993).

FIG. 11.: Reproducibility of the Empirical Descriptor (Rat Heart).

This graph represents the mean obtained during calculations of the empirical descriptor in the heart, in the Wistar rat, as a function of time. The values of the descriptor are shown on the y-axis. The x-axis comprises 2 columns. The column on the left corresponds to the first determination of the descriptor performed. The column on right corresponds to the second determination of the descriptor performed. A gap of 7 days separates the two measurements.

No significant deviation is observed between the two columns.

FIG. 12.: Sensitivity of the Empirical Descriptor (Rat Heart).

This graph represents the mean obtained during the calculations of empirical descriptor in the heart, in the Zucker rat, with or without treatment with Rosiglitazone or with a placebo. The values of the descriptor are shown on the y-axis. The x-axis comprises 4 columns: the two on the left and the two on the right; the two on the left relate to the values observed for the rats at the start of observation before any injection, and the two on the right relate to the rats 21 days after injection. The white columns indicate rats having undergone an injection of Rosiglitazone, the black columns indicate rats having undergone an injection of a placebo.

No significant deviation is observed between the first and second columns.

No significant deviation is observed between the second and fourth columns.

A significant deviation is observed between the first and third columns (p=0.033).

A significant deviation is observed between the third and fourth columns (p=0.006).

FIG. 13.: Correlation Between the Empirical Index, and the GIR (Rat Heart).

FIG. 13 represents the correlation between the empirical glucose transport indices in the cardiac cells and the GIR (Glucose Infusion Rate) measured by euglycaemic hyper-insulinaemic clamp. The values of the empirical index are shown on the x-axis, and the GIR values (mg/kg/min) on the y-axis. Each point corresponds to one Wistar rat. The measurement protocol was repeated for each rat, the GIR and the empirical index determined for each rat, then a dot designating the rat is plotted in FIG. 13 as a function of its index and its GIR. The solid straight line represents the regression line of the all of the rats (y=2.02*x+13.99; R²=0.47 (R=0.688); p=0.019).

FIG. 14.: Correlation Between the Empirical Index, and the GIR (Rat Skeletal Muscle).

FIG. 14 represents the correlation between the empirical glucose transport indices in the skeletal muscle cells and the GIR (Glucose Infusion Rate) measured by euglycaemic hyperinsulinaemic clamp. The values of the empirical index are shown on the x-axis, and the GIR values (mg/kg/min) on the y-axis. Each point corresponds to one Wistar rat. The measurement protocol was repeated for each rat, the GIR and the empirical index determined for each rat, then a dot designating the rat is plotted in FIG. 14 as a function of its index and its GIR. The solid straight line represents the regression line of the all of the rats (y=2.1316*x+16.858, R²=0.5032, p=0.022).

FIG. 15.: Reproducibility of the Empirical Descriptor (Rat Skeletal Muscle).

This graph represents the mean obtained during calculations of the empirical descriptor in the skeletal muscle, in the Wistar rat, as a function of time. The values of the descriptor are shown on the y-axis. The x-axis comprises 2 columns. The column on the left corresponds to the first determination of the descriptor performed. The column on the right corresponds to the second determination of the descriptor performed. A gap of 7 days separates the two measurements.

No significant deviation is observed between the two columns.

FIG. 16.: Sensitivity of the Empirical Descriptor (Rat Skeletal Muscle).

This graph represents the mean obtained during calculations of the empirical descriptor in the skeletal muscle, in the Zucker rat, with or without treatment with Rosiglitazone or with a placebo. The values of the descriptor are shown on the y-axis. The x-axis comprises 4 columns: the two on the left and the two on the right; the two on the left relate to the values observed for the rats at the start of observation before any injection, and the two on the right relate to the rats 21 days after injection. The white columns indicate rats having undergone an injection of Rosiglitazone, the black columns indicate rats having undergone an injection of a placebo.

No significant deviation is observed between the first and second columns.

No significant deviation is observed between the second and fourth columns.

A significant deviation is observed between the first and third columns (p=0.021).

A significant deviation is observed between the third and fourth columns (p=0.005).

FIG. 17.: Reproducibility of the Theoretical Index (Rat Heart)

This graph represents the variations obtained during calculations of the theoretical index of glucose transfer into cardiac cells in the Wistar rat. The values of the index are shown on the y-axis. The x-axis comprises 2 series of measurements 7 days apart. The value on the left corresponds to the first determination of the index performed. The value on the right corresponds to the second determination of the index performed.

No significant deviation is observed between the two series of measurements.

FIG. 18.: Sensitivity of the Theoretical Index (Rat Heart).

This graph represents the mean obtained during calculations of the theoretical index in the heart, in the Zucker rat, with or without treatment with Rosiglitazone or with a placebo. The values of the index are shown on the y-axis. The x-axis comprises 4 columns: the two on the left and the two on the right; the two on the left relate to the values observed for the rats at the start of observation before any injection, and the two on the right relate to the rats 7 days after injection. The white columns indicate rats having undergone an injection of Rosiglitazone, the black columns indicate rats having undergone an injection of a placebo.

A significant deviation is observed between the third and fourth columns (p<0.05).

FIG. 19.: Variation in the Coefficients k(2,1) for the Rat (Heart)

This graph represents the variations obtained during calculations of k(2,1), namely the coefficients of glucose transfer into cardiac cells. The values of k(2,1) are shown on the y-axis. The x-axis indicates whether the values were recorded in the “basal” state that is to say without injection of insulin (column on left), or in the “insulin” state that is to say after injection of insulin (column on right). The continuous lines link the white dots which mark the values obtained for Wistar rats (healthy), and the dashed lines link the black dots which mark the values obtained for the Zucker rats (insulin resistant).

The isolated white dot to the right of the column representing the measurements in the “insulin” state indicates the mean value measured in the “insulin” state for the Wistar rats and the standard deviation with a discrimination of p<0.05.

The isolated black dot to the right of the column representing the measurements in the “insulin” state indicates the mean value measured in the “insulin” state for the Zucker rats and the standard deviation.

FIG. 20.: Glycaemia after 14 Hours Fasting in the Rat

FIG. 20 represents the glycaemia of four groups of rats. The glycaemia is indicated in g/L along the y-axis. The four groups of rats are indicated by the four columns on the x-axis. The columns are considered from left to right. The control groups do not undergo any surgical operation.

The first column represents a control group of 35 individuals. The glycaemia of this group is 0.779+/−0.045 g/L after 14 hours fasting. This measurement is performed 5 days before the surgical operation.

The second column represents a control group of 11 individuals. The glycaemia of this group is 0.849+/−0.315 g/L after 14 hours fasting. This measurement is performed 7 days after the surgical operation.

The third column represents a group of “Sham” rats (rats having undergone the surgical operation, but without occlusion) of 13 individuals. The glycaemia of this group is 0.840+/−0.041 g/L after 14 hours fasting. This measurement is performed 7 days after the surgical operation.

The fourth column represents a group of “RI” rats (rats having undergone the surgical operation, and the occlusion) of 11 individuals. The glycaemia of this group is 0.806+/−0.052 g/L after 14 hours fasting. This measurement is performed 7 days after the surgical operation.

FIG. 21.: Post-Infarction Insulin Resistance (Rat Heart).

FIG. 21 represents the indices of cardiac insulin resistance (K(2,1)ᵢₙₛᵤₗᵢₙ/K(2,1)_{Basal}) (see paragraph II-2) of three groups of rats. The value of the index of cardiac insulin resistance is indicated along the y-axis. The three groups of rats are indicated by the three columns on the x-axis. The columns are considered from left to right. The measurements are carried out 7 days after the surgical operation. The control group did not undergo any surgical operation.

An index of insulin resistance equal to 1 signifies a total absence of response to insulin.

The first column represents a control group of 11 individuals. The index of cardiac insulin resistance of this group is 2.46+/−0.25.

The second column represents a group of “Sham” rats (rats having undergone the surgical operation, but without occlusion) of 13 individuals. The index of cardiac insulin resistance of this group is 1.62+/−0.16. This index is significantly different from the index observed for the control group (P<0.01)

The third column represents a group of “RI” rats (rats having undergone the surgical operation, and the occlusion) of 11 individuals. The index of cardiac insulin resistance of this group is 1.09+/−0.04. This index is significantly different from the index observed for the control group (P<0.01) and from the index observed for the “Sham” group (P<0.01).

### EXPERIMENTAL PART

### I Data Acquisition

### 1) Rat (Muscle)

The rat is operated on after general anaesthesia with sodium pentobarbital (60 mg/kg, intraperitoneal) and a first catheter was placed in the femoral vein. A second catheter was inserted into the adjacent artery. All the injections were carried out via the venous catheter and the blood samples taken via the arterial catheter. Throughout the experiment, the temperature of the rat is monitored and stabilised by means of a heating blanket regulated by a system connected to a rectal probe continuously measuring the internal temperature of the rat (“Homeothermic blanket control unit”, Harvard Apparatus, UK).

The rat receives a first bolus of 6-DIG labelled with iodine 123 (about 250 μCi, this activity being counted in a Capintec CRC15R ionisation chamber supplied by Aries, France) in the basal condition. The activity of the 6-DIG is monitored with the gamma camera (field of view 10 cm, intrinsic resolution 1.8 mm, Biospace, France), or with the NaI probe (Scintibloc from Crismatec (Nemours, France) and ScintiSPEC, Aries) in skeletal muscle cells (quadriceps of hind paw) for 20 minutes. During these 20 minutes, the radioactivity is counted in “LIST” mode (TD. Cradduck, Computers in Nuclear Medicine, Vol. 5; No. 1, 1985, Radiographics) and analysed a posteriori

The rat then receives an injection of insulin (2.5 IU/kg), performed 5 minutes before a second injection of the tracer (about 250 μCi). The activity of the 6-DIG is again monitored with the gamma camera or with the NaI probe in skeletal muscle cells for 20 minutes. During these 20 minutes, the radioactivity is counted in “LIST” mode and analysed a posteriori.

### 2) Rat (Heart)

The rat is operated on after general anaesthesia with sodium pentobarbital (60 mg/kg, intraperitoneal) and a first catheter was placed in the femoral vein. A second catheter was inserted into the adjacent artery. All the injections were carried out via the venous catheter and the blood samples taken via the arterial catheter. Throughout the experiment, the temperature of the rat is monitored and stabilised by means of a heating blanket regulated by a system connected to a rectal probe continuously measuring the internal temperature of the rat (“Homeothermic blanket control unit”, Harvard Apparatus, UK).

The rat receives a first bolus of 6-DIG labelled with iodine 123 (about 250 μCi, this activity being counted in a Capintec CRC15R ionisation chamber supplied by Aries, France) in the basal condition. The activity of the 6-DIG is monitored with the gamma camera (field of view 10 cm, intrinsic resolution 1.8 mm, Biospace, France) in cardiac cells for 20 minutes. During these 20 minutes, the radioactivity is counted in “LIST” mode and analysed a posteriori.

The rat then receives an injection of insulin (2.5 IU/kg), performed 5 minutes before a second injection of the tracer (about 250 μCi). The activity of the 6-DIG is again monitored with the gamma camera in cardiac cells for 20 minutes. During these 20 minutes, the radioactivity is counted in “LIST” mode and analysed a posteriori.

### 3) Rat (Heart and Muscle)

The rat is operated on after general anaesthesia with sodium pentobarbital (60 mg/kg, intra-peritoneal) and a first catheter was placed in the femoral vein. A second catheter was inserted into the adjacent artery. All the injections were carried out via the venous catheter and the blood samples taken via the arterial catheter. Throughout the experiment, the temperature of the rat is monitored and stabilised by means of a heating blanket regulated by a system connected to a rectal probe continuously measuring the internal temperature of the rat (“Homeothermic blanket control unit”, Harvard Apparatus, UK).

The rat receives a first bolus of 6-DIG labelled with iodine 123 (about 250 μCi, this activity being counted in a Capintec CRC15R ionisation chamber supplied by Aries, France) in the basal condition. The activity of the 6-DIG is monitored with the gamma camera or the NaI probe in skeletal muscle cells (quadriceps of hind paw) and in cardiac cells for 20 minutes. During these 20 minutes, the radioactivity is counted for both sets of cells in “LIST” mode and analysed a posteriori.

The rat then receives an injection of insulin (2.5 IU/kg), performed 5 minutes before a second injection of the tracer (about 250 μCi). The activity of the 6-DIG is again monitored with the gamma camera and the NaI probe in skeletal muscle cells and cardiac cells for 20 minutes. During these 20 minutes, the radioactivity is counted in “LIST” mode and analysed a posteriori.

### Measurements in Man

The patient must be fasting, preferably for at least 8 hours.

In man, it is impossible to inject a bolus of insulin as is done in the rat. The decrease in glycaemia induced by the insulin must be carried out much more gradually, over 10 to 15 minutes.

The measurement protocol in man is adapted from a validated protocol (Erturk E et al., Clin Endocrinol Metab, 1998, 83: 2350-2354; Nye E J et al., J Neurol Endocrinol, 2001, 13:524-530.), used in clinical practice for the simultaneous evaluation of the corticotropic and somatotropic axes.

Contraindications to Performing the Test:

The patient must preferably not be older than 65 years, there must be no epileptic risk (personal history of epilepsy, cranial trauma, upper route pituitary surgery), there must be no history of coronary disease or history of cardiac arrhythmia, and the patient must not be a pregnant woman.

Monitoring:

Medical attendance is necessary from the start of the assay.

Attention is drawn to the need to watch for clinical signs relating to hypoglycaemia (the percentages correspond to the frequency of occurrence of the symptoms):

sweating (63%), hunger (50%), palpitations (51%), tremor (31%), loss of conscience, convulsions (<3%)

Somnolence is common, and must be combatted.

In case of occurrence of these symptoms:
a glycaemia assay is performed,
if the hypoglycaemia is confirmed, the sugar restoration process is implemented.

### Process for Sugar Restoration:

if the patient is conscious, the sugar restoration is carried out orally, for example by means of 3 sugar lumps with water or a carton of orange juice,
in case of loss of consciousness: glucose 30%:2 ampoules strictly by i.v. route or Glucagon (Glucagen®): 1 mg by i.v., i.m. or subcutaneous route,
monitoring of the glycaemia to be resumed 15 to 20 minutes after sugar restoration. If the hypoglycaemia persists, the sugar restoration process must be repeated.

### 4) Man (Muscle)

The patient receives a first intravenous injection of 6-DIG (2.5 mCi) labelled with iodine 123 under the basal conditions. A NaI probe (Scintibloc de Crismatec (Nemours, France) and ScintiSPEC, Aries) is placed on the patient's thigh muscle, and the variation in labelled 6-DIG in the cells of the thigh is observed for 20 minutes. The radioactivity is recorded in “LIST” mode and analysed a posteriori.

The patient then receives an injection of insulin (0.1 IU/kg), this dose can be adjusted on the basis of the patient's body mass index (BMI) (0.1 IU/kg if BMI<25 kg/m², and 0.15 U/kg if BMI>25 kg/m²), then 10 minutes later a second injection of 6-DIG (2.5 mCi) labelled with iodine 123. The variation in 6-DIG in the cells of the thigh is observed by means of the NaI probe for 20 minutes, and the radioactivity is recorded in “LIST” mode and analysed a posteriori.

### 5) Man (Heart)

The patient receives a first intravenous injection of 6-DIG (2.5 mCi) labelled with iodine 123 under the basal conditions.

He is placed under a gamma camera (Symbia T2, Siemens) and the observation is made at the thoracic level to measure the variation in labelled 6-DIG in the cardiac cells, for 20 minutes post-injection, and the radioactivity is recorded in “LIST” mode and analysed a posteriori. The patient then receives an injection of insulin (0.1 IU/kg), this dose can be adjusted on the basis of the patient's body mass index (BMI) (0.1 IU/kg if BMI<25 kg/m², and 0.15 U/kg if BMI>25 kg/m²), then 10 minutes later a second injection of 6-DIG (2.5 mCi) labelled with iodine 123. He is again placed under a gamma camera, and the observation of the cardiac cells is resumed for 20 minutes post-injection, and the radioactivity is recorded in “LIST” mode and analysed a posteriori.

### 6) Man (Heart and Muscle)

The patient receives a first intravenous injection of 6-DIG (2.5 mCi) labelled with iodine 123 under the basal conditions. He is placed under a gamma camera and the observation is made at the thoracic level to measure the variation in labelled 6-DIG in the cardiac cells, for 20 minutes post-injection, the radioactivity is recorded in “LIST” mode and analysed a posteriori. In parallel, a NaI probe is placed on the patient's thigh muscle, and the variation in labelled 6-DIG in the cells of the thigh is observed for 20 minutes, the radioactivity is recorded in “LIST” mode and analysed a posteriori.

The patient then receives an injection of insulin (0.1 IU/kg), this dose can be adjusted on the basis of the patient's body mass index (BMI) (0.1 IU/kg if BMI<25 kg/m², and 0.15 U/kg if BMI>25 kg/m²), then 10 minutes later a second injection of 6-DIG (2.5 mCi) labelled with iodine 123. He is again placed under a gamma camera, and the observation of the cardiac cells is resumed for 20 minutes, the radioactivity is recorded in “LIST” mode and analysed a posteriori. In parallel, the variation in 6-DIG in the cells of the thigh is observed by means of the NaI probe for 20 minutes, and the radioactivity is recorded in “LIST” mode and analysed a posteriori.

### 7) Man (Blood Samples)

The patient receives a first intravenous injection of 6-DIG (2.5 mCi) labelled with iodine 123 under the basal conditions. Blood samples are taken by means of a catheter for 20 minutes at the times t=0 and 30 s, and 1, 2, 5, 10, 15 and 20 minutes.

The patient then receives an injection of insulin (0.1 IU/kg) then 10 minutes later a second injection of 6-DIG (2.5 mCi) labelled with iodine 123. A second series of blood samples are taken by means of a catheter during 20 minutes at the times t=0 and 30 s, and 1, 2, 5, 10, 15 and 20 minutes.

The quantity of 6-DIG in the samples taken is determined by means of a gamma counter (COBRA II, Packard), the samples have volumes of 1 mL, and the radioactivity is recorded for 30 seconds per sample.

### 8) Man (Blood In Vivo)

The patient receives a first intravenous injection of 6-DIG (2.5 mCi) labelled with iodine 123 under the basal conditions. He is placed under a gamma camera and the observation is made at the level of the aortic arch to measure the variation in labelled 6-DIG in the blood, for 20 minutes post-injection and the radioactivity recorded in “LIST” mode and analysed a posteriori.

The patient then receives an injection of insulin (0.1 IU/kg), then 10 minutes later a second injection of 6-DIG (2.5 mCi) labelled with iodine 123. He is again placed under a gamma camera and the observation is made at the level of the aortic arch to measure the variation in labelled 6-DIG in the blood, for 20 minutes post-injection and the radioactivity is recorded in “LIST” mode and analysed a posteriori.

### II Mathematical Model

The biological data previously obtained (see I: Data Acquisition), are combined with a mathematical model adapted for each set of data depending on the muscle observed. The sets of data obtained on the blood do not require a specific mathematical model, and they can be processed by the mathematical models applied to the skeletal muscle or to the heart.

These models enable the calculation of an index characterizing the rate of glucose transport depending on the set of cells observed.

This index is equal to the ratio of the coefficient of fractional transfer of the 6DIG from the blood compartment towards the “tissue” compartment in presence of insulin, to that obtained in the basal state. It is correlated with insulin resistance.

This coefficient of fractional transfer of the 6DIG from the blood compartment towards the “tissue” compartment corresponds to the kinetics of transfer of the 6DIG from the blood compartment towards the “tissue” compartment.

Two mathematical models make it possible to obtain the kinetics depending on the set of cells observed:

### 1) Kinetics for Muscle

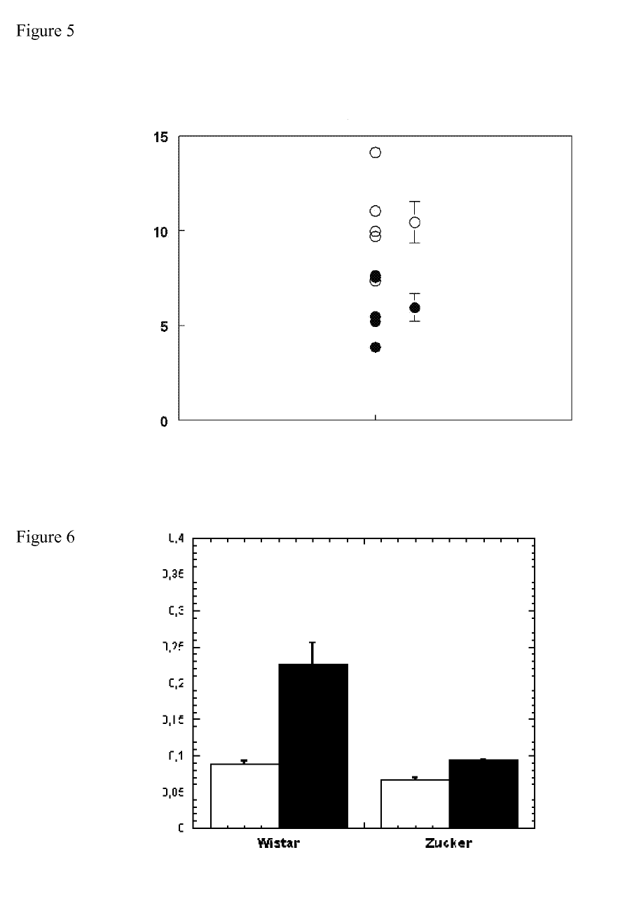

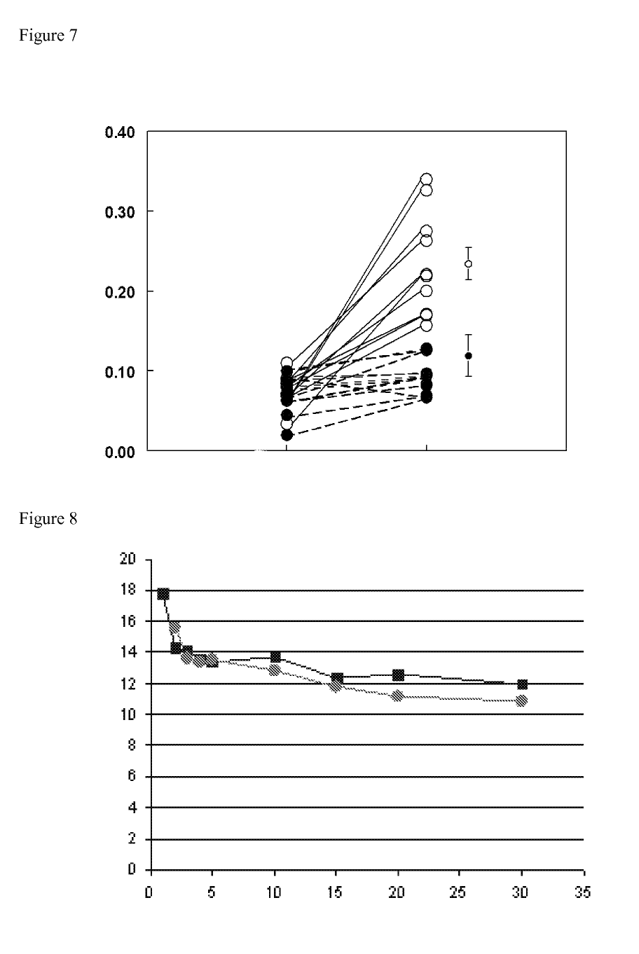

When the cells observed are skeletal muscle cells, the coefficient of transfer (k3, FIG. 2) is calculated using the following model:

The biological data are transposed into a 3-compartment mathematical model, based on that used by Bertoldo et al. for the measurement of the transport of 3OMG under euglycaemic hyperinsulinaemic clamp in man [Bertoldo A. et al., J. Clin. Endocrinol. Metab., 2005, 90(3), 1752-1759]. This model makes it possible, from a measurement performed on the skeletal muscle, to distinguish the interstitial compartment from the intracellular compartment, which corresponds to the muscle cell. We are concerned with k3, which is the constant of entry into the intracellular compartment, to determine the index characterizing the rate of glucose transport.

The model comprises 4 kinetic parameters and can be described mathematically by the following system of equations:

Ċᵢ(t)=k₁Cₚ(t)−(k₂+k₃)Cᵢ(t)+k₄Cₑ(t) Cᵢ(0)=0

Ċₑ(t)=k₃Cᵢ(t)−k₄Cₑ(t) Cₑ(0)=0

C(t)=(1−V_{b})(Cᵢ(t)+Cₑ(t))+V_{b}Cₚ(t)

With Cₚ (FIG. 2) which represents the concentration of [¹²³I]6-DIG in the arterial plasma, Cᵢ is the extracellular concentration of [¹²³I]6-DIG normalised to the volume of the tissue, Cₑ is the concentration of [¹²³I]6-DIG in the tissue, C is the total concentration of the activity of the iodine 123 measured in the region of interest (ROI), k₁ and k₂ are the exchange parameters between the plasma and the extracellular space, and k₃ and k₄ are the constants for transport entering and leaving the cell. V_{b} is the fraction occupied by the total blood volume in the region of interest.

### 2) Kinetics for the Heart

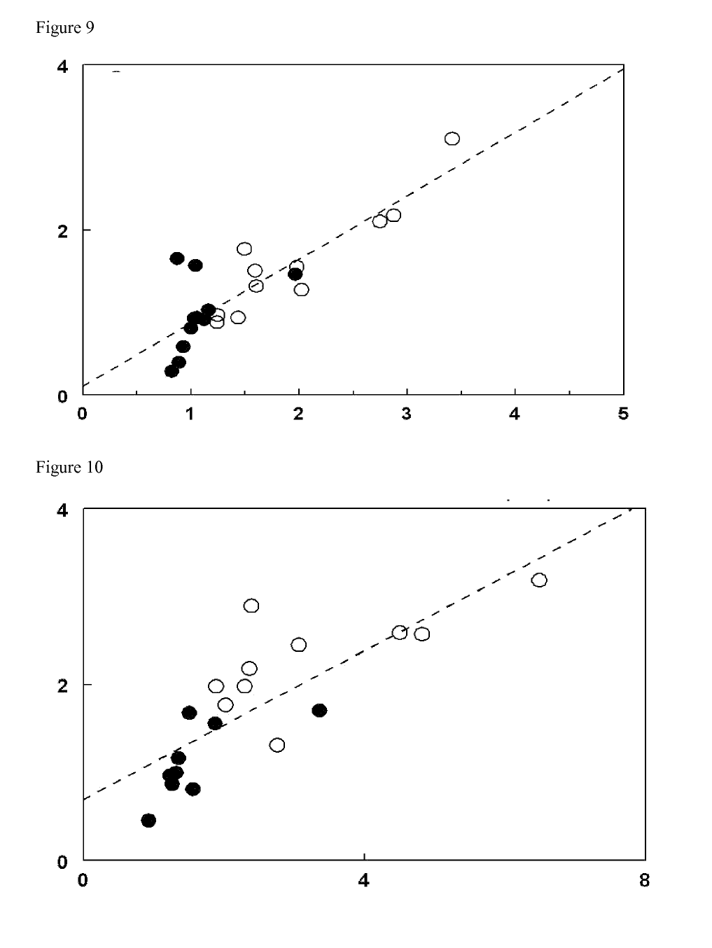

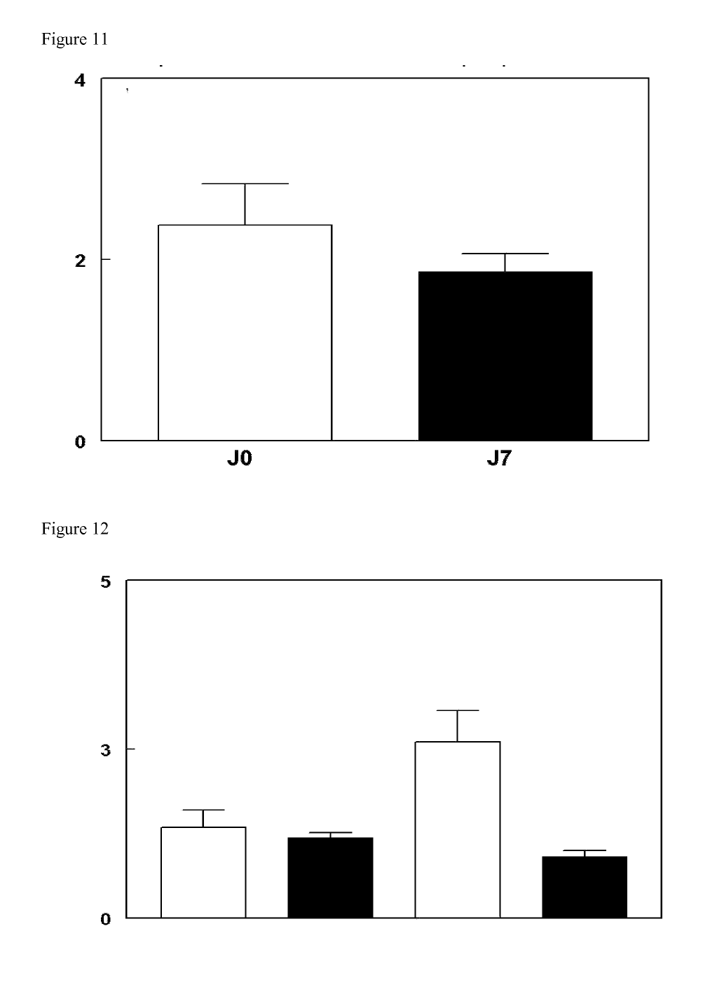

When the cells observed are cardiac cells, the coefficient of transfer (k_{2,1}, FIG. 3) is calculated using the following model:

The model utilised is a three-compartment mammillary model, derived from that used to measure the kinetic parameters of transport of 3OMG, the reference tracer for glucose transport (Cobelli, 1989; Am. J. Physiol., 257, E444-E450). It makes it possible to study the biological behaviour of the tracer after injection in the rat in vivo (Slimani L. et al., CR Biol, 2002, 325(4), 529-546). The central compartment (s1) represents the plasma. It is into this compartment that the 6DIG is injected and from which an irreversible outflow occurs: k(0.1) (Jacquez, 1972; “Compartmental analysis in Biology and Medicine, Ed. Elsevier, New-York 1972). Bidirectional exchanges take place between this central compartment and the compartments 2 and 3, respectively representing the heart and the totality of the other organs. The radioactivity measured at the level of compartments 1 and 2 is designated respectively as q1 and q2.

The exchanges between the compartments are assumed to be linear. This is justified by the fact that the 6DIG is used at very low concentrations, which are negligible relative to its Km. In glucose membrane transport kinetics of the Michaëlis type, the coefficients of transfer kᵢⱼ, the quantity of tracer q, the Michaëlis constant Km and the maximum rate of transport Vm are connected by the relationship:

$$kij = \frac{Vmi}{Kmi + qi} and kji = \frac{Vmj}{Kmj + qj}$$

Since qi<<Kmi and qj<<Kmj, a linear approximation of the model can be envisaged. In this case, the transfer coefficients are given by:

$$kij = \frac{Vm}{Km}$$

Thus, assuming that the model is linear and the system in the stationary state, the equations governing the compartmental exchanges are:

$$\frac{d {q}^{1} ( t )}{d t} = - ( {\sum }_{j = 2}^{3} {k}_{j 1} + {k}_{01} ) {q}_{1} ( t ) + {\sum }_{j = 2}^{3} {k}_{1 j} {q}_{j} ( t ) + {u}_{1} ( t )$$

$$\frac{d {q}_{j} ( t )}{d t} = {k}_{j 1} {q}_{1} ( t ) - {k}_{1 j} {q}_{j} ( t )$$

$$j = 2 , 3$$

where kᵢⱼ is the parameter representing the fractional transfer of the tracer from the compartment j to the compartment i (j≠i), q is the quantity of tracer in the compartment in question and u(t) is the injection function.

### III Empirical Descriptor

Three stages are necessary to validate an empirical descriptor: select a descriptor, compare the correlation between the results obtained with that descriptor and those obtained with the mathematical model, and finally validate the discriminating power of the empirical descriptor between a healthy patient and a patient suffering from insulin resistance.

### 1) Selection of an Empirical Descriptor

It has been found that the following empirical descriptor:

Activity [(10 min insulin×20 min insulin)/(10 min basal×20 min basal)]×[Ratio (50-90)×Ratio (900-1200)]

is appropriate in the implementation of the process of the invention.

In this equation, activity signifies the number of counts derived from the degradation of the iodine 123 recorded at a given moment in the data acquisition. “10 min” and “20 min” correspond respectively to the sum of the counts recorded during a defined time of X seconds, which time varies from 1 to 30 seconds and in particular 10 seconds, immediately preceding the 10^{th} minute, and the 20^{th} minute, after injection of the 6DIG. “Insulin” signifies that the activity is recorded after previous injection of insulin, and “basal” signifies that the activity is recorded without previous injection of insulin.

Ratio indicates that a ratio of slopes is being considered. The two slopes considered for establishing the ratio are those in the “basal” condition and in the “insulin” condition. The slopes represent the variation in the number of counts derived from the degradation of the iodine 123 recorded over an interval of time. The ratio is thus calculated by dividing the variation in the “insulin” condition by the variation in the “basal” condition. The interval of time during which the variation is measured is indicated in seconds from the injection of 6DIG.

Ratio 50-90 signifies that the ratio considered relates to the variations in number of counts recorded, in the “basal” condition and in the “insulin” condition, between the 50^{th} second and the 90^{th} second after injection of 6DIG.

Similarly, ratio 900-1200 signifies that the ratio considered relates to the variations in number of counts recorded, in the “basal” condition and in the “insulin” condition, between the 900^{th} second and the 1200^{th} second after injection of 6DIG.

### 2) Determination of Correlation

Heart

From a set of data obtained on 22 rats (11 Wistar rats (healthy) and 11 Zucker rats (insulin resistant)), the theoretical glucose transport index into the heart (calculated from the mathematical model) and the empirical glucose transport index into the heart (calculated by means of the descriptor described above) are determined. For each of the rats, the results for these two indices are plotted in FIG. 9. The straight line represents all of the rats and the correlation obtained is r²=0.73. The empirical descriptor is judged to be satisfactory.

Skeletal Muscle

From a set of data obtained on 19 rats (10 Wistar rats (healthy) and 9 Zucker rats (insulin resistant)), the theoretical glucose transport index into the skeletal muscle (calculated from the mathematical model) and the empirical glucose transport index into the skeletal muscle (calculated by means of the descriptor described above) are determined. For each of the rats, the results for these two indices are plotted in FIG. 10. The straight line represents all of the rats and the correlation obtained is r²=0.63. The empirical descriptor is judged to be satisfactory.

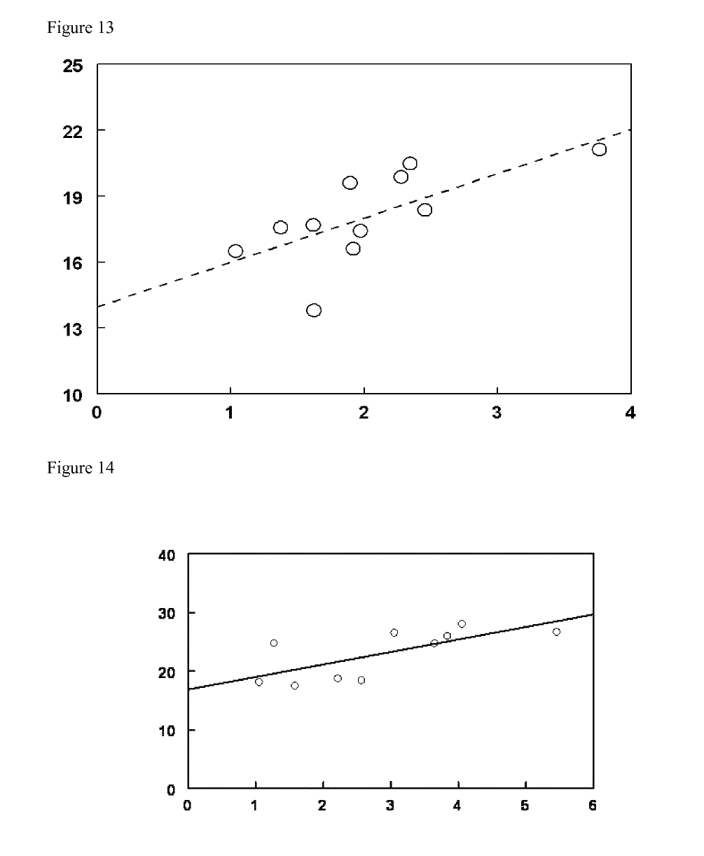

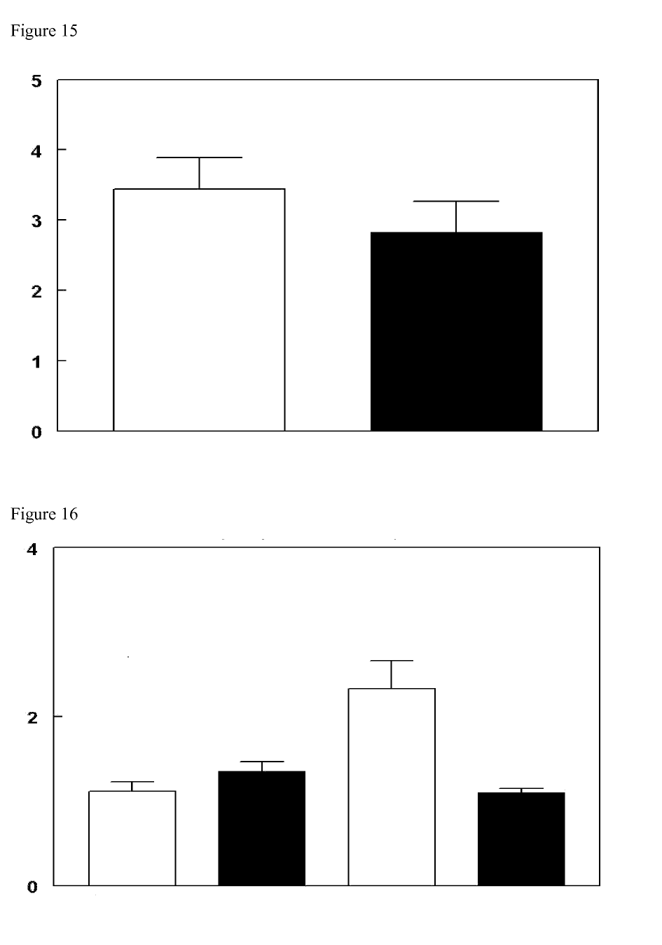

From a set of data obtained on 14 rats (7 Wistar rats (healthy) and 7 Zucker rats (insulin resistant)), the theoretical glucose transport index into the skeletal muscle (calculated from the mathematical model) and the empirical glucose transport index into the skeletal muscle (calculated by means of the descriptor described above) are determined. For each of the rats, the results for these two indices are plotted in FIG. 4. The straight line represents all of the rats and the correlation obtained is r²=0.76 (significance p=0.001). The empirical descriptor is thus judged to be satisfactory. It makes it possible to shorten the acquisitions to 20 minutes. In fact, in 20 minutes all the data necessary for the calculation of the empirical glucose transport index are collected, and it becomes possible to determine the insulin resistance.

### 3) Validation of the Descriptor

Application of the empirical descriptor to rats under duplicate conditions 20 minutes.

A first bolus of 6DIG (˜1.4 MBq) is injected followed by 20 minutes of “basal” acquisition. At the end of the “basal” acquisition, a bolus of insulin is injected (2.5 IU/kg). Five minutes later, a second bolus of 6DIG (˜1.4 MBq) is injected, according to the same protocol as during the “basal” condition, followed by 20 minutes of acquisition of the radioactivity in the “insulin” condition.

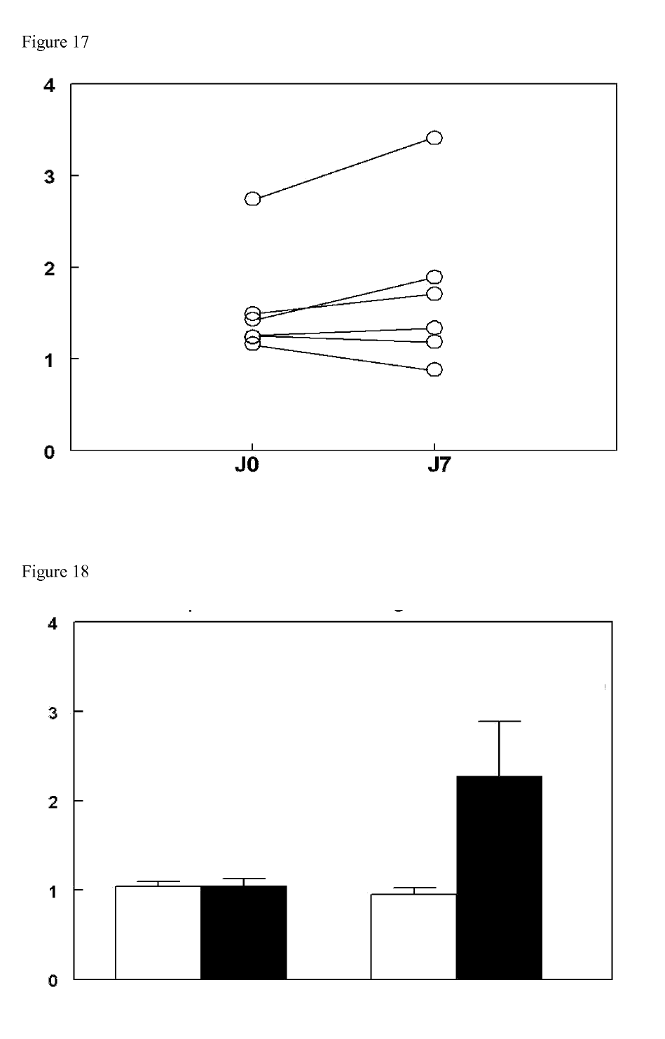

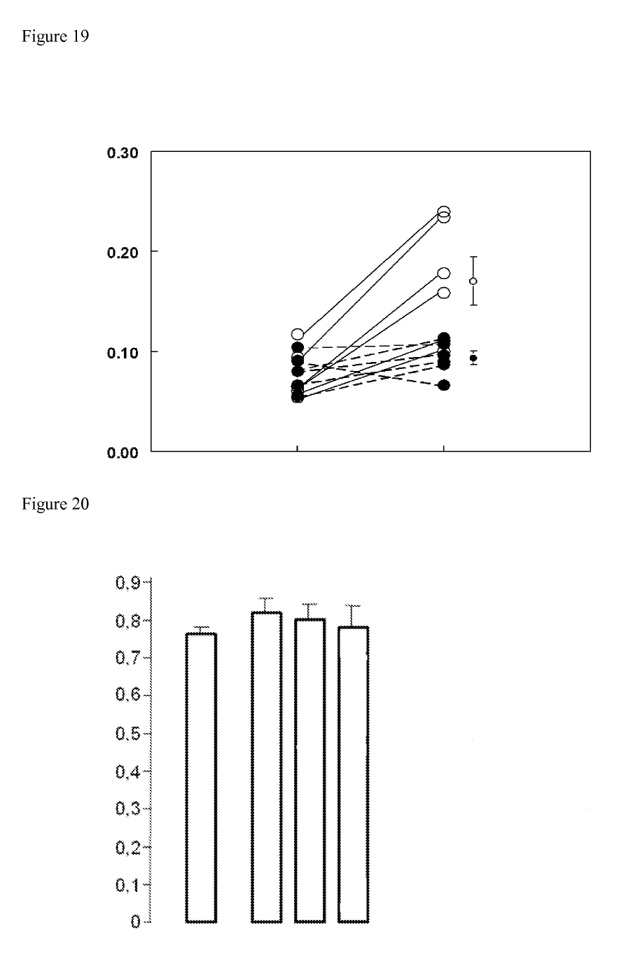

The data recorded are processed with the empirical descriptor described above, and the results for the empirical glucose transport indices are presented in FIG. 5.

It is found that the descriptor makes it possible to separate the Wistar rats (healthy) from the Zucker rats (insulin resistant) significantly (p=0.012), in the context of a short protocol of 45 minutes in total.

Heart

Reproducibility Heart

The empirical index characterizing the rate of glucose transport into the heart was determined twice, 7 days apart, on the same animal. This characterization was reproduced on 8 Wistar rats (healthy).

FIG. 11 represents the results obtained. Reproducibility of the empirical index characterizing the rate of glucose transport into the heart, over time, is observed.

Sensitivity Heart

The empirical index characterizing the rate of glucose transport into the heart was determined twice, 21 days apart, on the same animal, treated with Rosiglitazone (6 animals) or a placebo (6 animals). The first measurement is performed before treatment (Rosiglitazone or placebo) and the second after treatment. The animals are Zucker rats (insulin resistant). FIG. 12 represents the results obtained. A similar index value is observed before treatment. Three weeks after treatment with Rosiglitazone or with a placebo, the value of the empirical descriptor of the rats treated with the Rosiglitazone is significantly greater (p=0.006) than that of the empirical descriptor of the rats treated with a placebo, and the value of the empirical descriptor of the rats treated with Rosiglitazone is significantly greater (p=0.033) than that of the empirical descriptor of the same rats before treatment with Rosiglitazone.

Empirical Descriptor Heart Compared to the Euglycaemic Hyperinsulinaemic Clamp

The empirical index characterizing the rate of glucose transport into the heart was compared to the results obtained by means of the reference technique in the measurement of insulin resistance, the euglycaemic hyperinsulinaemic clamp. The empirical index character—using the rate of glucose transport into the heart, and the GIR (Glucose Infusion Rate) that is to say the general sensitivity of the subject to insulin measured by means of the clamp are determined for 11 Wistar rats. The GIR and the empirical index are determined 7 days apart, and the GIR is determined first.

FIG. 13 represents the results obtained. A significant correlation (p=0.019) between the empirical index characterizing the rate of glucose transport into the heart and the GIR is observed.

The empirical index characterizing the rate of glucose transport into the heart is sensitive, reproducible, and provides results comparable to the euglycaemic hyper-insulinaemic clamp.

Skeletal Muscle

Reproducibility Skeletal Muscle

The empirical index characterizing the rate of glucose transport into the skeletal muscle was determined twice, 7 days apart, on the same animal. This characterization was reproduced on 8 Wistar rats (healthy).

FIG. 15 represents the results obtained. Reproducibility of the empirical index characterizing the rate of glucose transport into the skeletal muscle, over time, is observed.

Sensitivity Skeletal Muscle

The empirical index characterizing the rate of glucose transport into the skeletal muscle was determined twice, 21 days apart, on the same animal, treated with Rosiglitazone (6 animals) or a placebo (6 animals). The first measurement is performed before treatment (Rosiglitazone or placebo) and the second after treatment. The animals are Zucker rats (insulin resistant).

FIG. 16 represents the results obtained. A similar index value is observed before treatment. Three weeks after treatment with Rosiglitazone or with a placebo, the value of the empirical descriptor of the rats treated with the Rosiglitazone is significantly greater (p=0.005) than that empirical descriptor of the rats treated with a placebo, and the value of the empirical descriptor of the rats treated with Rosiglitazone is significantly greater (p=0.021) than that of the empirical descriptor of the same rats before treatment with Rosiglitazone.

Empirical Descriptor Skeletal Muscle Compared to the Euglycaemic Hyperinsulinaemic Clamp

The empirical index characterizing the rate of glucose transport into the skeletal muscle was compared to the results obtained by means of the reference technique in the measurement of insulin resistance, the euglycaemic hyperinsulinaemic clamp. The empirical index characterizing the rate of glucose transport into the skeletal muscle, and the GIR (Glucose Infusion Rate) that is to say the general sensitivity of the subject to insulin measured by means of the clamp are determined for 11 Wistar rats. The GIR and the empirical index are determined 7 days apart, and the GIR is determined first.

FIG. 14 represents the results obtained. A significant correlation (p=0.022) between the empirical index characterizing the rate of glucose transport into the skeletal muscle and the GIR is observed.

The empirical index characterizing the rate of glucose transport into the skeletal muscle is sensitive, reproducible, and provides results comparable to the euglycaemic hyperinsulinaemic clamp.

### IV Examples

In order to exemplify the method described in the present invention, two strains of rat were utilised, a healthy rat strain (Wistar) and an insulin resistant strain (Zucker).

### 1) Determination of Insulin Resistance in the Rat, by Measurement on Skeletal Muscle Cells.

The data acquisition protocol is followed as described in paragraph I-1, and the mathematical model applied to determine the index characterizing the rate of glucose transport is as described in paragraph II-1.

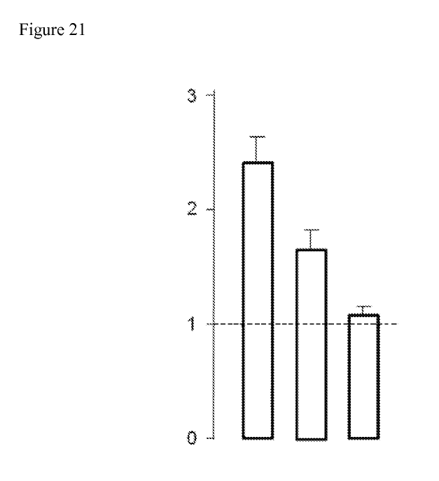

FIG. 6 represents the means of the k3 obtained in the conditions referred to as “basal” and “insulin”: the rise in the coefficients for entry of 6DIG into the intracellular compartment under insulin is appreciably greater in the healthy rats (Wistar) than in the insulin resistant rats (Zucker).

FIG. 7 represents the change in the k3 obtained case by case in the conditions referred to as “basal” and “insulin”: the Wistar rats have a coefficient for entry of 6DIG under insulin generally greater than the Zucker rats. The index characterizing the rate of glucose transport is calculated by the ratio k3 insulin/k3 basal. The indices obtained in the skeletal muscle are greater in the Wistar rats than in the Zucker rats.

The 6DIG measurement protocol and the mathematical model make it possible to detect a defect in glucose transport into the skeletal muscle cells of the insulin resistant rats (Zucker).

### 2) Determination of Insulin Resistance in the Rat, by Measurement on Cardiac Cells.

The data acquisition protocol is followed as described in paragraph I-2, and the mathematical model applied to determine the index characterizing the rate of glucose transport is as described in paragraph II-2.

FIG. 19 represents the means of the k(2,1) obtained in the conditions referred to as “basal” and “insulin”. The rise in the coefficients for entry of 6DIG in the cardiac cells under insulin is appreciably greater in the healthy rats (Wistar) than in the insulin resistant rats (Zucker).

Reproducibility

The index characterizing the rate of glucose transport into the heart was measured twice, 7 days apart, on the same animal. This measurement was reproduced on 6 Wistar rats (healthy).

FIG. 17 represents the results obtained. Reproducibility of the measurement over time is observed.

Sensitivity

The index characterizing the rate of glucose transport into the heart was measured twice, 7 days apart, on the same animal, treated with Rosiglitazone or a placebo. The first measurement is performed before treatment (Rosiglitazone or placebo) and the second after treatment. The animals are Zucker rats (insulin resistant).

FIG. 18 represents the results obtained. Similar index values are observed before treatment. Seven days after treatment with Rosiglitazone or a placebo, the index values of the rats treated with Rosiglitazone are significantly greater (p<0.05) than those of the rats treated with a placebo.

The measurement of the index characterizing the rate of glucose transport into the heart is sensitive and reproducible.

### 3) Determination of Insulin Resistance in the Rat, by Measurement on Skeletal Muscle and Cardiac Cells.

The data acquisition protocol is followed as described in paragraph I-3, and the mathematical models applied to determine the indices characterizing the rates of glucose transport are as described in paragraphs II-1 and II-2, for the data collected respectively on the muscle and the heart.

### 4) Determination of Post-Myocardial Infarction Insulin Resistance in the Rat

Material and Methods

a—Thoracic Surgery

The myocardial infarction experimental model utilised here is that induced by temporary in situ ligature of the left coronary artery. This protocol induces a transmural infarction, the scale of which is sufficient to lead to the development of severe cardiac insufficiency. The animals are anaesthetised by intraperitoneal injection of a 1:1 mixture of ketamine/xylazine, (50 mg/kg and 10 mg/kg respectively), and a volatile maintenance anaesthesia (isoflurane) is applied until the end of the surgical protocol. The rats are intubated and artificially ventilated (0.1 mL/100 g body weight, frequency: 65/min, gas mixture: 80% air+19.5% oxygen+0.5% isoflurane). Throughout the process, the rat is located on a heating blanket (Homeothermic Blanket System, Harvard Apparatus) making it possible to maintain its body temperature at 37° C. A skin incision is performed to the left of the sternum and the pectoral muscles are parted to effect a thoracotomy by opening of the fourth intercostal space. The heart is externalised by pressure on the thoracic cage. A ligature thread (Softsilk 5/0) is passed under the left coronary artery, at the height of the apex of the auricle, then the heart is reintegrated into the thoracic cage. After 5 min of stabilisation, the ligature threads are passed into a catheter gently inserted into the cavity until substantial resistance is felt; the catheter is maintained in this position by means of a clamp for 1 hr, thus ensuring the occlusion of the left coronary artery (“RI” rats for ischaemia reperfusion). After one hour of ischaemia, the ligature is lifted by removing the clamp, and the muscle layers resutured after having reconstituted the pleural cavity by pressure on the thoracic cage, then the volatile anaesthesia is stopped. The “Sham” rats undergo the same surgical process, but the occlusion is not performed. Finally, a group of “Control” rats does not undergo any surgical protocol.

b—Measurement of Glycaemia

The glycaemia is measured on all the animals, 5 days before the surgical protocol (J−5) and 7 days after the intervention (J+7). After a fasting period of 14±1 hours, the alert animals are placed in a restraint tube. One drop of blood is taken by incision of the distal part of the tail. The glycaemia is determined by means of reactive strips (Accu-Chek® Performa, Laboratoires Roche) read by an auto-controller (Accu-Chek® Performa, Laboratoires Roche).

c—Measurement of Insulin Resistance by Nuclear Imaging

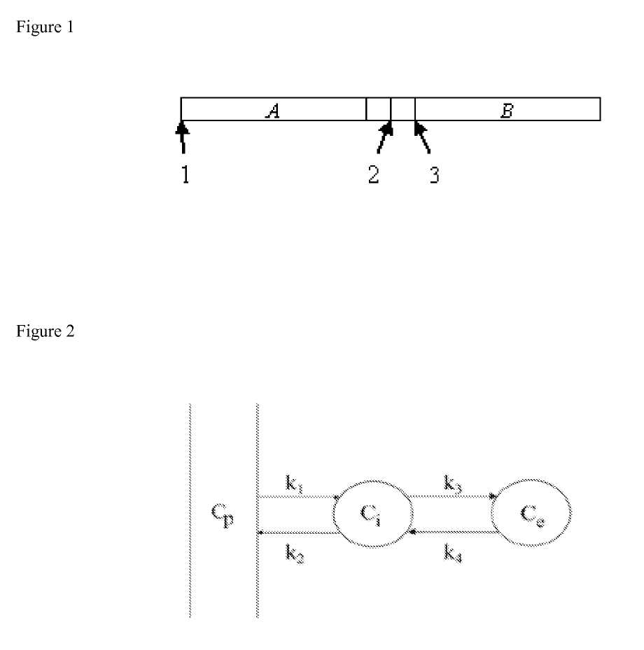

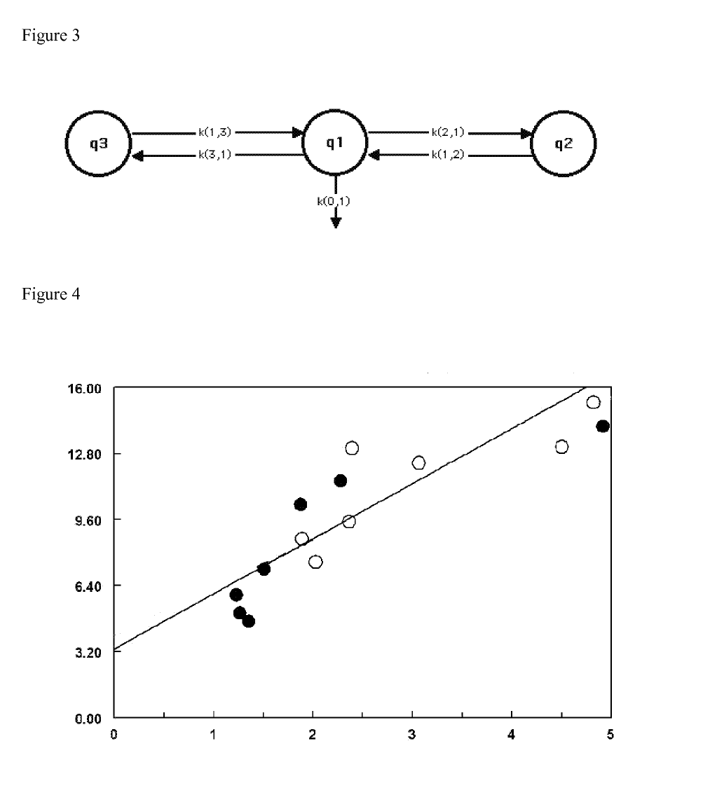

Seven days after the thoracic surgery, each rat is anaesthetised by intra-peritoneal injection of a solution of sodium pentothal (54.7 mg/ml). A polyurethane catheter is placed in the animal's left carotid, and another in the right jugular vein. The catheters, filled with heparinized physiological serum solution, are then “tunnelised” and fixed on the back of the animal. The rat is then placed under a dedicated γ camera for small animals (Biospace, France) connected to a computer acquisition system. The arterial glycaemia of the rat is measured by means of reactive strips (Accu-Chek® Performa, Laboratoires Roche). A bolus of 170 μCi of 6DIG is injected into the jugular vein the rinsed with 100 μL of heparinized NaCl. About 20 μL of arterial blood are taken after the start of the acquisition, at each of the following times: 1, 2, 3, 4, 5, 6, 7, 9, 11, 13, 15 and 20 minutes (FIG. 1, measurement A).

After the first 20 minutes of acquisition, which constitute the test in the basal state (FIG. 1, measurement A), a bolus of insulin (Actrapid, Novo Nordisk, France, 7.5 IU/mL, 100 μL per 300 g of body weight) is injected (FIG. 1, time 2). After 5 minutes (FIG. 1, time 3), the glycaemia is measured and a bolus of about 170 μCi of 6DIG is injected. Blood samples are collected at the same times as during the test in the basal state (FIG. 1, measurement B). The blood samples are passed into a counter (Cobra II Auto-Gamma, Packard, Canberra Company) which counts the radioactivity in counts per minute, to determine the blood kinetics of the tracer.

At the end of the protocol the rat is euthanized by an intravenous injection (1 mL) of saturated KCl. The heart is then removed, sponged and weighed on a precision balance (Sartorius, France). The activity of this organ is counted (Cobra II Auto-Gamma, Packard, Canberra Company). The images obtained during the 6DIG protocol are analysed by means of the software provided by Biospace (γ-acquisition, γ-vision+). From activity data, SAAMII computer software (Simulation, Analys and Modeling Institute, Seattle, Wash., 1997, USA), based on the compartmental model described in paragraph II-2 and represented in FIG. 3, provides a quantitative index of the cardiac insulin resistance of the animal through the ratio k(2,1)_{Insulin}/k(2,1)_{Basal} (FIG. 3).

Results

a—Glycaemia

The fasting glycaemia is statistically equivalent in all the experimental groups 7 days after the surgical protocol (FIG. 20).

b—Post-Cardiac Infarction Insulin Resistance

The cardiac insulin sensitivity is reduced by a factor of 2 in the Sham group compared to the controls, suggesting that the surgical protocol, in itself, constitutes a cardiac stress which alters insulin sensitivity (FIG. 21).

The heart of the animals of the RI group is practically totally resistant to insulin, 7 days after the surgical protocol, suggesting that myocardial infarction affects the sensitivity of the myocardium to insulin, independently of the stress connected with the surgical protocol.

These results show that myocardial infraction in the rat induces a marked phenomenon of cardiac insulin resistance, 7 days after the experimental ischaemia, that is to say at the time of the temporary overexpression of leptin and myocardial pro-inflammatory cytokines. Moreover, this total absence of response of the myocardium to insulin is not accompanied by any significant change in the glycaemia, which suggests that the insulin resistance observed is limited to the myocardium, without the other insulin sensitive tissues being affected.

### 5) Determination of Insulin Resistance in Man, by Measurement on Skeletal Muscle Cells.

The data acquisition protocol is followed as described in paragraph I-4, and the mathematical model applied to determine the index characterizing the rate of glucose transport is as described in paragraph II-1.

### 6) Determination of Insulin Resistance in Man, by Measurement on Cardiac Cells.

The data acquisition protocol is followed as described in paragraph I-5, and the mathematical model applied to determine the index characterizing the rate of glucose transport is as described in paragraph II-2.

### 7) Determination of Insulin Resistance in Man, by Measurement on Skeletal Muscle and Cardiac Cells.

The data acquisition protocol is followed as described in paragraph I-6, and the mathematical models applied to determine the indices characterizing the rates of glucose transport are as described in paragraphs II-1 and II-2, for the data collected on the muscle and the heart respectively.

### 8) Determination of Insulin Resistance in Man, by Measurement on Blood Samples.

The data acquisition protocol is followed as described in paragraph I-7, this measurement is performed at the same time as data acquisition as described in paragraphs I-4, 5 or 6; and the mathematical model applied to determine the index characterizing the rate of glucose transport corresponds to that described in paragraph II-2 if the measurement is performed in addition to a measurement on cardiac cells, or to that described in paragraph II-1 if the measurement is performed in addition to a measurement on skeletal muscle cells.

### 9) Determination of Insulin Resistance in Man, by Measurement on the Blood In Vivo.

The data acquisition protocol is followed as described in paragraph I-8, this measurement is performed at the same time as data acquisition as described in paragraphs I-4, 5 or 6; and the mathematical model applied to determine the index characterizing the rate of glucose transport corresponds to that described in paragraph II-2 if the measurement is performed in addition to a measurement on cardiac cells, or to that described in paragraph II-1 if the measurement is performed in addition to a measurement on skeletal muscle cells.

### 10) Determination of Insulin Resistance in the Dog, by Measurement on Cardiac Cells.

Animals: male beagle dogs aged about 1 year, weighing between 15 and 20 kg and fed with a standard diet Jape 21 (Ets L. Pietrement).

The dogs, premedicated with an intramuscular injection of ketamine (10 mg/kg), are anaesthetised by means of an intravenous injection of sodium thiopental (25 mg/kg). The animals are intubated and ventilated throughout the experiment and kept asleep with halothane in the ventilation circuit (oxygen-enriched air).

6DIG (20 μmol) labelled with iodine 123 (about 4 mCi) is injected intravenously. The scintigraphy images are acquired by means of a standard gamma camera equipped with a high resolution collimator and the spectrophotometry is set at 160 keV with a 20% window. The variation in the thoracic radioactivity (heart, lungs and liver) with time is measured for 30 minutes post-injection, at the rate of one image per minute. The change in the blood activity is obtained by taking blood samples (1, 2, 3, 4, 5, 10. 15, 20 and 30 min p.i.) and the measurement of the radioactivity by means of a gamma counter (Packard).

Two protocols were followed, the first was implemented in fasting animals (withdrawal of food the night before the experiment, n=1) and the second was implemented in animals perfused with a GIK (Glucose/Insulin/Potassium) solution containing in particular insulin (30% glucose, 4 g/l of KCl and 80 IU/kg of insulin, n=2) and maintained throughout the experiment.

The activity is greatest in the liver at 5 minutes but decreases rapidly. The pulmonary activity is low and comparable to the background noise which makes it possible to image the heart effectively. The blood activity decreases very rapidly in the blood as observed in either the rat or the mouse.

The slope measured between 2 and 5 minutes for the dog under GIK is 0.044 (0.022 for the other dog under GIK) and only 0.013 when fasting (FIG. 8).

1-28. (canceled)

29. A method for the determination of insulin resistance in a mammal comprising at least one glucose derivative, halogenated in the 6 position, previously administrated to said mammal and measurement, thanks to γ radiation detection:
on the one hand, of the variation in the quantity (as a function of time) of the aforesaid derivative in muscle cells during a given period Δt, after administration of the aforesaid derivative, and
on the other hand, of the variation in quantity (as a function of time) of the aforesaid derivative in the aforesaid muscle cells during a period essentially equal to the aforesaid period Δt, after administration of the aforesaid derivative, preceded by an administration of insulin.

30. A method for the determination of insulin resistance in a man comprising at least one glucose derivative, halogenated in the 6 position, previously administrated to said man and measurement, thanks to γ radiation detection:
on the one hand, of the variation in the quantity (as a function of time) of the aforesaid derivative in muscle cells during a given period Δt, after administration of the aforesaid derivative, and
on the other hand, of the variation in quantity (as a function of time) of the aforesaid derivative in the aforesaid muscle cells during a period essentially equal to the aforesaid period Δt, after administration of the aforesaid derivative, preceded by an administration of insulin.

31. The method according to claim 29, in which the muscle cells are skeletal muscle cells.

32. The method according to claim 29, in which the muscle cells are cardiac cells.

33. The method according to claim 29, in which the muscle cells are skeletal muscle cells and cardiac cells.

34. The method according to claim 29 for the determination of insulin resistance in the rat, in which the muscle cells are skeletal muscle cells

35. The method according to claim 29 for the determination of insulin resistance in the rat, in which the muscle cells are cardiac cells.

36. The method according to claim 29 for the determination of insulin resistance in the rat, in which the muscle cells are skeletal muscle cells and cardiac cells.

37. The method according to claim 30 for the determination of insulin resistance in man, in which the muscle cells are selected from skeletal muscle cells.

38. The method according to claim 30 for the determination of insulin resistance in man, in which the muscle cells are selected from cardiac cells.

39. The method according to claim 30 for the determination of insulin resistance in man, in which the muscle cells are selected from skeletal muscle cells and cardiac cells.

40. The method according to claim 29, in which the glucose derivative halogenated in the 6 position is a 6-deoxy-6-halogeno-glucose, the said glucose derivative halogenated in the 6 position being a pure tracer of glucose transport.

41. The method according to claim 40, in which the 6-deoxy-6-halogeno-glucose is the 6-deoxy-6-iodoglucose.

42. The method according to claim 40, in which the 6-deoxy-6-halogeno-glucose is the 6-deoxy-6-fluoroglucose.

43. The method according to claim 29, in which the 6-deoxy-6-iodoglucose is labelled with a radioactive isotope of iodine.

44. The method according to claim 29, in which the 6-deoxy-6-iodoglucose is labelled with iodine 123.

45. The method according to claim 37 for the determination of insulin resistance in man, in which the halogenated derivative of glucose is 6-deoxy-6-iodoglucose.

46. The method according to claim 37 for the determination of insulin resistance in man, in which the halogenated derivative of glucose is 6-deoxy-6-fluoroglucose.

47. The method according to claim 38 for the determination of insulin resistance in man, in which the halogenated derivative of glucose is 6-deoxy-6-iodoglucose.

48. The method according to claim 38 for the determination of insulin resistance in man, in which the halogenated derivative of glucose is 6-deoxy-6-fluoroglucose.

49. The method according to claim 39 for the determination of insulin resistance in man, in which the halogenated derivative of glucose is 6-deoxy-6-iodoglucose.

50. The method according to claim 39 for the determination of insulin resistance in man, in which the halogenated derivative of glucose is 6-deoxy-6-fluoroglucose.

51. Process for the determination of insulin resistance in a mammal capable of exhibiting insulin resistance by detection of γ radiation comprising:
a first stage of measurement of the variation in the quantity (as a function of time) of a glucose derivative halogenated in the 6 position, previously administered to the mammal, which measurement takes place in muscle cells and optionally the blood of the said mammal during a given period Δt, by means for detection of γ radiation, to establish a first group of data;
a second stage of measurement of the variation in the quantity (as a function of time) of the aforesaid glucose derivative halogenated in the 6 position, previously administered, following an administration of insulin, to the mammal, which measurement takes place in muscle cells and optionally the blood of the said mammal, during a period essentially equal to the aforesaid period Δt, by means for detection of γ radiation, to establish a second group of data;
a third stage of calculation of an index characterizing the rate of glucose transport, the said glucose transport taking place from the blood or the interstitial compartment towards the muscle cells, and the said index being capable of being determined by means of a mathematical algorithm and/or an empirical descriptor, from the aforesaid two groups of data;
a fourth stage of comparison of the aforesaid index characterizing the rate of glucose transport with the index characterizing the rate of glucose transport obtained in a healthy mammal by implementing in the said healthy mammal the three stages defined above with regard to the mammal the said comparison making it possible to determine a deviation which can be associated with insulin resistance in the said mammal.

52. Process for the determination of insulin resistance in a patient capable of exhibiting insulin resistance by detection of γ radiation comprising:
a first stage of measurement of the variation in the quantity (as a function of time) of a glucose derivative halogenated in the 6 position, previously administered to the patient, which measurement takes place in muscle cells and optionally the blood of the said patient, during a given period Δt, by means for detection of γ radiation, to establish a first group of data;
a second stage of measurement of the variation in the quantity (as a function of time) of the aforesaid glucose derivative halogenated in the 6 position, previously administered, following an administration of insulin, to the patient, which measurement takes place in muscle cells and optionally the blood of the said patient, during a period essentially equal to the aforesaid period Δt, by means for detection of γ radiation, to establish a second group of data;
a third stage of calculation of an index characterizing the rate of glucose transport, the said glucose transport taking place from the blood or the interstitial compartment towards the muscle cells, and the said index being capable of being determined by means of a mathematical algorithm and/or an empirical descriptor, from the aforesaid two groups of data;
a fourth stage of comparison of the aforesaid index characterizing the rate of glucose transport with the index characterizing the rate of glucose transport obtained in a healthy patient, by implementing in the said healthy patient, the three stages defined above with regard to the patient, the said comparison making it possible to determine a deviation which can be associated with insulin resistance in the said patient.

53. The process according to claim 51, in which the index calculated from a mathematical algorithm is the theoretical index, the said theoretical index corresponding in particular to the ratio of the kinetics of glucose transport taking place from the blood or the interstitial compartment towards the muscle cells.

54. The process for the determination of insulin resistance according to claim 52, in a patient capable of exhibiting insulin resistance, in which the muscle cells are skeletal muscle cells, and comprising a third stage of calculation of an index characterizing the rate of glucose transport, the said glucose transport taking place from the interstitial compartment towards the skeletal muscle cells, and the said index being obtained by a mathematical algorithm or an empirical descriptor from the aforesaid two groups of data.

55. The process for the determination of insulin resistance according to claim 52, in a patient capable of exhibiting insulin resistance, in which the muscle cells are cardiac cells, and comprising a third stage of calculation of an index characterizing the rate of glucose transport, the said glucose transport taking place from the blood towards the cardiac cells, and the said kinetics being obtained by a mathematical algorithm or an empirical descriptor from the aforesaid two groups of data;

56. The process for the determination of insulin resistance according to claim 52, in a patient capable of exhibiting insulin resistance, in which the muscle cells are skeletal muscle cells and cardiac cells, and comprising:
a first stage comprising:
a measurement of the variation in the quantity (as a function of time) of a glucose derivative halogenated in the 6 position, previously administered to the patient, which measurement takes place in skeletal muscle cells of the said patient, to establish a first group of data relating to the skeletal muscle, and
a measurement of the variation in the quantity (as a function of time) of a glucose derivative halogenated in the 6 position, previously administered to the patient, which measurement takes place in cardiac cells of the said patient, to establish a first group of data relating to the heart,
a second stage comprising:
a measurement of the variation in the quantity (as a function of time) of a glucose derivative halogenated in the 6 position, previously administered after an administration of insulin, to the patient, which measurement takes place in skeletal muscle cells of the said patient, to establish a second group of data relating to the skeletal muscle, and
a measurement of the variation in the quantity (as a function of time) of a glucose derivative halogenated in the 6 position, previously administered after an administration of insulin, to the patient, which measurement takes place in cardiac cells of the said patient, to establish a second group of data relating to the heart,
a third stage of calculation comprising
the calculation of an index characterizing the rate of glucose transport, the said glucose transport taking place from the interstitial compartment towards the skeletal muscle cells, and the said index being capable of being determined by means of a mathematical algorithm and/or an empirical descriptor from the aforesaid two groups of data relating to the skeletal muscle, and
the calculation of an index characterizing the rate of glucose transport, the said glucose transport taking place from the blood towards the cardiac cells, and the said index being capable of being determined by means of a mathematical algorithm and/or an empirical descriptor from the aforesaid two groups of data relating to the heart;
a fourth stage of comparison
of the aforesaid index characterizing the rate of glucose transport, into the skeletal muscle cells, with the index characterizing the rate of glucose transport, into the skeletal muscle cells, obtained in a healthy patient, by implementing in the said healthy patient the three stages as defined above, the deviation making it possible to characterize the insulin resistance of the said patient.
of the aforesaid index characterizing the rate of glucose transport, into the cardiac cells, with the index characterizing the rate of glucose transport, into the cardiac cells, obtained in a healthy patient, by implementing in the said healthy patient the three stages as defined above, the deviation making it possible to characterize the insulin resistance of the said patient.

57. The process for the determination of insulin resistance according to claim 51, in a mammal capable of exhibiting insulin resistance comprising:
a first stage, performed during a given period Δt, comprising
a measurement, by means for detection of γ radiation, of the variation in the quantity (as a function of time) of a glucose derivative halogenated in the 6 position, previously administered to the mammal which measurement takes place on blood samples from the said mammal, the said samples having been taken during the aforesaid given period Δt, to establish a first group of data relating to the blood, and
a measurement, by means for detection of γ radiation, of the variation in the quantity (as a function of time) of a glucose derivative halogenated in the 6 position, previously administered to the mammal, which measurement takes place in muscle cells of the said mammal, to establish a first group of data relating to the muscle;
a second stage, performed during a period essentially equal to the aforesaid period Δt, comprising
a measurement, by means for detection of γ radiation, of the variation in the quantity (as a function of time) of a glucose derivative halogenated in the 6 position, previously administered after an administration of insulin, to the mammal, which measurement takes place on blood samples from the said mammal, the said samples having been taken during the aforesaid given period Δt, to establish a second group of data relating to the blood, and
a measurement, by means for detection of γ radiation, of the variation in the quantity (as a function of time) of a glucose derivative halogenated in the 6 position, previously administered after an administration of insulin, to the mammal, which measurement takes place in muscle cells of the said mammal, to establish a second group of data relating to the muscle;
a third stage of calculation of an index characterizing the rate of glucose transport, the said glucose transport taking place from the blood towards the muscle cells, and the said index being determined by means of a mathematical algorithm, the calculation of this index making use of the groups of data relating to the blood, and the groups of data relating to the muscle,
a fourth stage of comparison of the aforesaid index characterizing the rate of glucose transport with the index characterizing the rate of glucose transport obtained in a healthy mammal, by implementing in the said healthy mammal, the three stages defined above with regard to the mammal, the said comparison making it possible to determine a deviation which can be associated with insulin resistance in the said mammal.

58. The process for the determination of insulin resistance according to claim 52, in a patient capable of exhibiting insulin resistance comprising:
a first stage, performed during a given period Δt, comprising
a measurement, by means for detection of γ radiation, of the variation in the quantity (as a function of time) of a glucose derivative halogenated in the 6 position, previously administered to the patient, which measurement takes place on blood samples from the said patient, the said samples having been taken during the aforesaid given period Δt, to establish a first group of data relating to the blood, and
a measurement, by means for detection of γ radiation, of the variation in the quantity (as a function of time) of a glucose derivative halogenated in the 6 position, previously administered to the patient, which measurement takes place in muscle cells of the said patient, to establish a first group of data relating to the muscle;
a second stage, performed during a period essentially equal to the aforesaid period Δt, comprising
a measurement, by means for detection of γ radiation, of the variation in the quantity (as a function of time) of a glucose derivative halogenated in the 6 position, previously administered after an administration of insulin, to the patient, which measurement takes place on blood samples from the said patient, the said samples having been taken during the aforesaid given period Δt, to establish a second group of data relating to the blood, and
a measurement, by means for detection of γ radiation, of the variation in the quantity (as a function of time) of a glucose derivative halogenated in the 6 position, previously administered after an administration of insulin, to the patient, which measurement takes place in muscle cells of the patient, to establish a second group of data relating to the muscle;
a third stage of calculation of an index characterizing the rate of glucose transport, the said glucose transport taking place from the blood towards the muscle cells, and the said index being determined by means of a mathematical algorithm, the calculation of this index making use of the groups of data relating to the blood, and the groups of data relating to the muscle,
a fourth stage of comparison of the aforesaid index characterizing the rate of glucose transport with the index characterizing the rate of glucose transport obtained in a healthy patient, by implementing in the said healthy patient, the three stages defined above with regard to the patient, the said comparison making it possible to determine a deviation which can be associated with insulin resistance in the said patient.

59. The process for the determination of insulin resistance according to claim 51, in a mammal capable of exhibiting insulin resistance comprising:
a first stage, performed during a given period Δt, comprising
a measurement, by means for detection of γ radiation, of the variation in the quantity (as a function of time) of a glucose derivative halogenated in the 6 position, previously administered to the mammal, which measurement takes place in muscle cells of the said mammal, to establish a first group of data relating to the muscle, and
a measurement, by means for detection of γ radiation, of the variation in the quantity (as a function of time) of a glucose derivative halogenated in the 6 position, previously administered to the mammal, which measurement takes place in the blood of the said mammal, to establish a first group of data relating to the blood,
a second stage, performed during a period essentially equal to the aforesaid period Δt, comprising
a measurement, by means for detection of γ radiation, of the variation in the quantity (as a function of time) of a glucose derivative halogenated in the 6 position, previously administered after an administration of insulin, to the mammal, which measurement takes place in muscle cells of the said mammal, to establish a second group of data relating to the muscle, and
a measurement, by means for detection of γ radiation, of the variation in the quantity (as a function of time) of a glucose derivative halogenated in the 6 position, previously administered after an administration of insulin, to the mammal, which measurement takes place in the blood of the said mammal, to establish a second group of data relating to the blood,
a third stage of calculation of an index characterizing the rate of glucose transport, the said glucose transport taking place from the blood towards the muscle cells, and the said index being determined by means of a mathematical algorithm, the calculation of this index making use of the groups of data relating to the blood, and the groups of data relating to the muscle,
a fourth stage of comparison of the aforesaid index characterizing the rate of glucose transport with the index characterizing the rate of glucose transport obtained in a healthy mammal, by implementing in the said healthy mammal the three stages defined above with regard to the mammal, the said comparison making it possible to determine a deviation which can be associated with insulin resistance in the said mammal.

60. The process for the determination of insulin resistance according to claim 52, in a patient capable of exhibiting insulin resistance comprising:
a first stage, performed during a given period Δt, comprising
a measurement, by means for detection of γ radiation, of the variation in the quantity (as a function of time) of a glucose derivative halogenated in the 6 position, previously administered to the patient, which measurement takes place in muscle cells of the said patient, to establish a first group of data relating to the muscle, and
a measurement, by means for detection of γ radiation, of the variation in the quantity (as a function of time) of a glucose derivative halogenated in the 6 position, previously administered to the patient, which measurement takes place in the blood of the said patient, to establish a first group of data relating to the blood,
a second stage, performed during a period essentially equal to the aforesaid period Δt, comprising
a measurement, by means for detection of γ radiation, of the variation in the quantity (as a function of time) of a glucose derivative halogenated in the 6 position, previously administered after an administration of insulin, to the patient, which measurement takes place in muscle cells of the said patient, to establish a second group of data relating to the muscle, and
a measurement, by means for detection of γ radiation, of the variation in the quantity (as a function of time) of a glucose derivative halogenated in the 6 position, previously administered after an administration of insulin, to the patient, which measurement takes place in the blood of the said patient, to establish a second group of data relating to the blood,
a third stage of calculation of an index characterizing the rate of glucose transport, the said glucose transport taking place from the blood towards the muscle cells, and the said index being determined by means of a mathematical algorithm, the calculation of this index making use of the groups of data relating to the blood, and the groups of data relating to the muscle,
a fourth stage of comparison of the aforesaid index characterizing the rate of glucose transport with the index characterizing the rate of glucose transport obtained in a healthy patient, by implementing in the said healthy patient, the three stages defined above with regard to the patient, the said comparison making it possible to determine a deviation which can be associated with insulin resistance in the said patient.

61. The process according to claim 51, in which the index calculated from an empirical descriptor is the empirical index, the said empirical descriptor itself being obtained from the aforesaid groups of data, by one or more mathematical operations in particular:
additions, subtractions, multiplications and divisions on the totality, a part or on parts of each of the two groups of data; and/or,
integrations and derivations on graphic representations, in particular curves, obtained from the totality, a part or on parts of each of the two groups of data.

62. The process according to claim 51, comprising the following stages making it possible to select an empirical descriptor:
1—determination of the theoretical index thanks to a mathematical algorithm, the said theoretical index then corresponding in particular to the ratio of the kinetics of glucose transport,
2—determination of a group of empirical descriptors making it possible to obtain a group of theoretical indices, each of the said empirical descriptors being obtained from the aforesaid two groups of data, by one or more mathematical operations;
3—comparison of the empirical indices with the theoretical index, in order to determine the empirical index closest to the theoretical index, and to select the empirical descriptor corresponding to that empirical index.

63. The process according to claim 51, in which the mammal is the rat and the cells are skeletal muscle cells.

64. The process according to claim 51, in which the mammal is the rat and the cells are skeletal muscle cells and cardiac cells.

65. The process according to claim 51, in which the 6 position is a 6-deoxy-6-halogeno-glucose is the 6-deoxy-6-fluoroglucose, the said glucose derivative halogenated in the 6 position being a pure tracer of glucose transport.

66. The process according to claim 51, in which the 6-deoxy-6-halogeno-glucose is the 6-deoxy-6-iodoglucose, the said glucose derivative halogenated in the 6 position being a pure tracer of glucose transport.

67. The process according to claim 66, in which the 6-deoxy-6-iodoglucose is labelled with a radioactive isotope of iodine.

68. The process according to claim 66, in which the 6-deoxy-6-iodoglucose is labelled with iodine 123.

69. The process for the determination of insulin resistance according to claim 54 in a patient capable of exhibiting insulin resistance in which the stages of measurements of the variation in the quantity of the aforesaid glucose derivative halogenated in the 6 position in skeletal muscle cells are carried out by means of a probe enabling the detection of γ radiation from iodine and from fluorine.

70. The process for the determination of insulin resistance according to claim 52 in a patient capable of exhibiting insulin resistance in which the stages of measurements of the variation in the quantity of the aforesaid glucose derivative halogenated in the 6 position in skeletal muscle cells are carried out by means of a NaI probe.

71. The process for the determination of insulin resistance according to claim 55, in a patient capable of exhibiting insulin resistance, in which the stages of measurements of the variation in the quantity of the aforesaid glucose derivative halogenated in the 6 position in cardiac cells are carried out using a means for detection of γ radiation from iodine and from fluorine.

72. The process for the determination of insulin resistance according to claim 55, in a patient capable of exhibiting insulin resistance, in which the stages of measurements of the variation in the quantity of the aforesaid glucose derivative halogenated in the 6 position in cardiac cells are carried out using a γ camera.

73. The process for the determination of insulin resistance according to claim 55, in a patient capable of exhibiting insulin resistance, in which the stages of measurements of the variation in the quantity of the aforesaid glucose derivative halogenated in the 6 position in skeletal muscle cells and cardiac cells are carried out by means of a probe enabling the detection of γ radiation from iodine and from fluorine, in particular a NaI probe for the skeletal muscle cells and a means for detection of γ radiation from iodine and from fluorine, in particular a γ camera for the cardiac cells.

74. The process for the determination of insulin resistance according to claim 58, in a patient capable of exhibiting insulin resistance, in which the stages of measurements of the variation in the quantity of the aforesaid glucose derivative halogenated in the 6 position in the blood are carried out using a means for detection of γ radiation from iodine and from fluorine.

75. The process for the determination of insulin resistance according to claim 52, in a patient capable of exhibiting insulin resistance, in which the stages of measurements of the variation in the quantity of the aforesaid glucose derivative halogenated in the 6 position in the blood are carried out using a γ camera.

76. The process for the determination of insulin resistance according to claim 58, in a patient capable of exhibiting insulin resistance, in which the stages of measurements of the variation in the quantity of the aforesaid glucose derivative halogenated in the 6 position in the blood are carried out using a gamma counter.

77. The process for the determination of insulin resistance according to claim 52, in a patient, comprising:
a first stage of measurement of the variation in the quantity (as a function of time) of 6-deoxy-6-iodoglucose previously injected into the patient, which measurement takes place in skeletal muscle cells of the said patient, during a given period Δt, in particular about 20 minutes, from the injection of 6-deoxy-6-iodoglucose, which measurement takes place by means of a probe in particular NaI enabling the detection of the γ radiation from iodine 123, to establish a first group of data;
a second stage of measurement of the variation in the quantity (as a function of time) of the 6-deoxy-6-iodoglucose previously injected, and preceded by an injection of insulin, in particular about 10 minutes before the injection of the aforesaid iodinated derivative into the patient, which measurement takes place in skeletal muscle cells of the said patient, during a given period Δt, in particular about 20 minutes, from the injection of 6-deoxy-6-iodoglucose, which measurement takes place by means of a probe in particular NaI enabling the detection of the y radiation from iodine 123, to establish a second group of data;
a third stage of calculation of an index characterizing the rate of glucose transport, the said glucose transport taking place from the interstitial compartment towards the skeletal muscle cells, and the said index being capable of being determined by means of a mathematical algorithm and/or an empirical descriptor, from the aforesaid two groups of data;
a fourth stage of comparison of the aforesaid index characterizing the rate of glucose transport with the index characterizing the rate of glucose transport obtained in a healthy patient, by implementing in the said healthy patient the three stages defined above with regard to the patient, the said comparison making it possible to determine a deviation which can be associated with insulin resistance of the said patient.

78. The process for the determination of insulin resistance according to claim 52, in a patient, comprising:
a first stage of measurement of the variation in the quantity (as a function of time) of 6-deoxy-6-iodoglucose previously injected into the patient, which measurement takes place in cardiac cells of the said patient, during a given period Δt, in particular about 20 minutes, from the injection of 6-deoxy-6-iodoglucose, which measurement takes place by means in particular of a γ camera enabling the detection of the γ radiation from iodine 123, to establish a first group of data;
a second stage of measurement of the variation in the quantity (as a function of time) of the 6-deoxy-6-iodoglucose previously injected, and preceded by an injection of insulin, in particular about 10 minutes before the injection of the aforesaid iodinated derivative into the patient, which measurement takes place in cardiac cells of the said patient, during a given period Δt, in particular about 20 minutes, which measurement takes place by means in particular of a γ camera enabling the detection of the γ radiation from iodine 123, to establish a second group of data;
a third stage of calculation of an index characterizing the rate of glucose transport, the said glucose transport taking place from the blood towards the cardiac cells, and the said index being capable of being determined by means of a mathematical algorithm and/or an empirical descriptor, from the aforesaid two groups of data;
a fourth stage of comparison of the aforesaid index characterizing the rate of glucose transport with the index characterizing the rate of glucose transport obtained in a healthy patient, by implementing in the said healthy patient the three stages defined above with regard to the patient, the said comparison making it possible to determine a deviation which can be associated with insulin resistance of the said patient.

79. Process for the determination of insulin resistance according to claim 52, in a patient, comprising:
a first stage comprising:
a measurement of the variation in the quantity (as a function of time) of 6-deoxy-6-iodoglucose previously injected into the patient, which measurement takes place in skeletal muscle cells of the said patient, during a given period Δt, in particular about 20 minutes, from the injection of 6-deoxy-6-iodoglucose, which measurement takes place by means of a probe in particular NaI enabling the detection of the γ radiation from iodine 123, to establish a first group of data relating to the skeletal muscle, and;
a measurement of the variation in the quantity (as a function of time) of 6-deoxy-6-iodoglucose previously injected into the patient, which measurement takes place in cardiac cells of the said patient, during a given period Δt, in particular about 20 minutes, from the injection of 6-deoxy-6-iodoglucose, which measurement takes place by means in particular of a γ camera enabling the detection of the γ radiation from iodine 123, to establish a first group of data relating to the heart;
a second stage comprising:
a measurement of the variation in the quantity (as a function of time) of the 6-deoxy-6-iodoglucose previously injected, and preceded by an injection of insulin, in particular about 10 minutes before the injection of the aforesaid iodinated derivative into the patient, which measurement takes place in skeletal muscle cells of the said patient, during a given period Δt, in particular about 20 minutes, from the injection of 6-deoxy-6-iodoglucose, which measurement takes place by means of a probe in particular NaI enabling the detection of the γ radiation from iodine 123, to establish a second group of data relating to the skeletal muscle, and;
a measurement of the variation in the quantity (as a function of time) of the 6-deoxy-6-iodoglucose previously injected, and preceded by an injection of insulin, in particular about 10 minutes before the injection of the aforesaid iodinated derivative into the patient, which measurement takes place in cardiac cells of the said patient, during a given period Δt, in particular about 20 minutes from the injection of 6-deoxy-6-iodoglucose, which measurement takes place by means in particular of a γ camera enabling the detection of the γ radiation from iodine 123, to establish a second group of data relating to the heart,
a third stage of calculation comprising:
the calculation of an index characterizing the rate of glucose transport, the said glucose transport taking place from the interstitial compartment towards the skeletal muscle cells, and the said index being capable of being determined by means of a mathematical algorithm and/or an empirical descriptor, from the aforesaid two groups of data relating to the skeletal muscle, and;
the calculation of an index characterizing the rate of glucose transport, the said glucose transport taking place from the blood towards the cardiac cells, and the said index being determined by means of a mathematical algorithm and/or an empirical descriptor, from the aforesaid two groups of data relating to the heart;
a fourth stage of comparison
of the aforesaid index characterizing the rate of glucose transport, into the skeletal muscle cells, with the index characterizing the rate of glucose transport, into the skeletal muscle cells obtained in a healthy patient, by implementing in the said healthy patient the three stages defined above with regard to the patient, the said comparison making it possible to determine a deviation which can be associated with insulin resistance of the said patient, and;
of the aforesaid index characterizing the rate of glucose transport, into the cardiac cells, with the index characterizing the rate of glucose transport, into the cardiac cells, obtained in a healthy patient, by implementing in the said healthy patient the three stages defined above with regard to the patient, the said comparison making it possible to determine a deviation which can be associated with insulin resistance of the said patient.

80. The process for the determination of insulin resistance according to claim 52, in a patient, comprising:
a first stage, performed during a given period Δt, comprising:
a measurement of the variation in the quantity (as a function of time) of 6-deoxy-6-iodoglucose previously injected into the patient, which measurement takes place on blood samples from the said patient, the said samples having been taken during the aforesaid given period Δt, in particular about 20 minutes, from the injection of 6-deoxy-6-iodoglucose, which measurement takes place by means of a gamma counter, enabling the detection of the γ radiation from iodine 123, to establish a first group of data relating to the blood, and
a measurement of the variation in the quantity (as a function of time) of 6-deoxy-6-iodoglucose previously injected into the patient, which measurement takes place in muscle cells of the said patient, during the aforesaid given period Δt, in particular about 20 minutes, from the injection of 6-deoxy-6-iodoglucose, which measurement takes place by means of a γ camera, or a NaI probe, enabling the detection of the γ radiation from iodine 123, to establish a first group of data relating to the muscle,
a second stage, performed during a period essentially equal to the aforesaid period Δt, comprising:
a measurement of the variation in the quantity (as a function of time) of 6-deoxy-6-iodoglucose previously injected, and preceded by an injection of insulin, in particular about 10 minutes before the injection of the aforesaid iodinated derivative into the patient, which measurement takes place on blood samples from the said patient, the said samples having been taken during the aforesaid given period Δt, in particular about 20 minutes, from the injection of 6-deoxy-6-iodoglucose, which measurement takes place by means of a gamma counter, enabling the detection of the γ radiation from iodine 123, to establish a second group of data relating to the blood, and
a measurement of the variation in the quantity (as a function of time) of 6-deoxy-6-iodoglucose previously injected, and preceded by an injection of insulin, in particular about 10 minutes before the injection of the aforesaid iodinated derivative into the patient, which measurement takes place in muscle cells of the said patient, during the aforesaid given period Δt, in particular about 20 minutes, from the injection of 6-deoxy-6-iodoglucose, which measurement takes place by means of a γ camera, or a NaI probe, enabling the detection of the γ radiation from iodine 123, to establish a second group of data relating to the muscle,
a third stage of calculation of an index characterizing the rate of glucose transport, the said glucose transport taking place from the blood towards the muscle cells, and the said index being determined by means of a mathematical algorithm, the calculation of this index making use of the groups of data relating to the blood, and the groups of data relating to the muscle,
a fourth stage of comparison of the aforesaid index characterizing the rate of glucose transport with the index characterizing the rate of glucose transport obtained in a healthy patient, by implementing in the said healthy patient the three stages defined above with regard to the patient, the said comparison making it possible to determine a deviation which can be associated with insulin resistance of the said patient.

81. The process for the determination of insulin resistance according to claim 52, in a patient, comprising:
a first stage, performed during a given period Δt, comprising:
a measurement of the variation in the quantity (as a function of time) of 6-deoxy-6-iodoglucose previously injected into the patient, which measurement takes place in the blood of the said patient, during the aforesaid given period Δt, in particular about 20 minutes, from the injection of 6-deoxy-6-iodoglucose, which measurement takes place by means of a γ camera, enabling the detection of the γ radiation from iodine 123, to establish a first group of data relating to the blood, and
a measurement of the variation in the quantity (as a function of time) of 6-deoxy-6-iodoglucose previously injected into the patient, which measurement takes place in muscle cells of the said patient, during the aforesaid given period Δt, in particular about 20 minutes, from the injection of 6-deoxy-6-iodoglucose, which measurement takes place by means of a γ camera, enabling the detection of the γ radiation from iodine 123, to establish a first group of data relating to the muscle,
a second stage, performed during a period essentially equal to the aforesaid period Δt, comprising:
a measurement of the variation in the quantity (as a function of time) of 6-deoxy-6-iodoglucose previously injected, and preceded by an injection of insulin, in particular about 10 minutes before the injection of the aforesaid iodinated derivative into the patient, which measurement takes place in the blood of the said patient, during the aforesaid given period Δt, in particular about 20 minutes, from the injection of 6-deoxy-6-iodoglucose, which measurement takes place by means of a γ camera, enabling the detection of the γ radiation from iodine 123, to establish a second group of data relating to the blood, and
a measurement of the variation in the quantity (as a function of time) of 6-deoxy-6-iodoglucose previously injected, and preceded by an injection of insulin, in particular about 10 minutes before the injection of the aforesaid iodinated derivative into the patient, which measurement takes place in muscle cells of the said patient, during the aforesaid given period Δt, in particular about 20 minutes, from the injection of 6-deoxy-6-iodoglucose, which measurement takes place by means of a γ camera, enabling the detection of the γ radiation from iodine 123, to establish a second group of data relating to the muscle,
a third stage of calculation of an index characterizing the rate of glucose transport, the said glucose transport taking place from the blood towards the muscle cells, and the said index being determined by means of a mathematical algorithm, the calculation of this index making use of the groups of data relating to the blood, and the groups of data relating to the muscle,
a fourth stage of comparison of the aforesaid index characterizing the rate of glucose transport with the index characterizing the rate of glucose transport obtained in a healthy patient, by implementing in the said healthy patient the three stages defined above with regard to the patient, the said comparison making it possible to determine a deviation which can be associated with insulin resistance of the said patient.
